(19) United States
(12) Patent Application Publication
(10) Pub. No.: US 2025/0376664 A1
(43) Pub. Date: Dec. 11, 2025

(54) Virus-Ribonucleoprotein Conjugates

(71) Applicant: Minghong Zhong, Sellersville, PA (US)

(72) Inventor: Minghong Zhong, Sellersville, PA (US)

(21) Appl. No.: 19/230,099

(22) Filed: Jun. 6, 2025

Related U.S. Application Data
(60) Provisional application No. 63/656,616, filed on Jun. 6, 2024.
(60) Provisional application No. 63/672,640, filed on Jul. 17, 2024.

(51) Int. Cl. C12N 7/00; C12N 9/12; C12N 9/22; C12N 15/11; C12N 15/86

(52) U.S. Cl. CPC C12N 7/00; C12N 9/1252; C12N 9/222; C12N 15/11; C12N 15/86; C07K 2319/80; C12N 2310/20; C12N 2750/14143; C12N 2750/14145; C12Y 207/07007

(57) ABSTRACT

Compositions of aCap virus-ribonucleic acid protein complex conjugates (VRC), wherein a viral capsid protein is covalently linked to a ribonucleic acid protein complex, i.e., a ribonucleoprotein (RNP), for selective delivery of the RNP into a targeted cell nucleus, and their preparation and uses as therapeutics.

### CROSS REFERENCE TO RELATED APPLICATIONS

The present application claims the benefits of U.S. Provisional Applications Ser. No. 63/656,616, filed on Jun. 6, 2024 and Ser. No. 63/672,640, filed on Jul. 17, 2024, the entire said inventions being incorporated herein by reference.

### INCORPORATION BY REFERENCE OF SEQUENCE LISTING

The contents of the sequence listing text named “Virus-Ribonucleoprotein Conjugates.xml”, which was created on Jun. 5, 2025, and 47,179 bytes in size, are incorporated herein by reference in its entirety.

### TECHNICAL FIELD OF THE INVENTION

The present invention relates to virus-ribonucleic acid protein complex conjugates (VRC), wherein a viral capsid protein is covalently linked to a ribonucleic acid protein complex, i.e., a ribonucleoprotein (RNP), for selective delivery of the RNP into a targeted cell nucleus. These VRCs include conjugates of RNA-guided DNA endonucleases such as clustered regularly interspaced short palindromic repeats (CRISPR)-Cas, the obligate mobile element-guided activity (OMEGA) system and Fanzors, and other RNA-guided gene-modifying RNPs.

In particular, the invention relates to compositions of a guide RNA-AAV conjugate for cell-targeted precise CRISPR gene editing wherein the guide RNA is covalently linked to an AAV capsid at its 5′-/3′-end, or its exposed nucleotide of an RNP or its non-nucleotide linker, its methods of preparation, and the uses of the conjugate as medicinal agents for treatment of viral infectious diseases and as gene regulation, replacement, disruption and/or correction-based therapeutics. The invention further relates to a composition comprising a guide RNA-AAV conjugate, a Cas protein or a Cas fusion protein with a DNA-directed DNA polymerase and a transgene or HDR template packaged in the AAV capsid, wherein the protein is bound to the guide RNA and optionally modified with one or more cis NLS for nuclear localization. The invention still further teaches a composition comprising a guide RNA-AAV conjugate and a transgene encoding a Cas protein-NLS packaged in the AAV capsid. In addition, the invention is adaptable to insert large transgenes by substitution of AAV with other viruses of appropriate capacities.

The invention further relates to compositions of a virus-CRISPR RNP conjugate linked by a flexible peptide chain between the C-terminus of a viral capsid protein and the N-terminus of a Cas protein which is optionally fused with a DNA-directed DNA polymerase at its C-terminus. The guide RNA of the VRC is bound to the Cas protein non-covalently.

### BACKGROUND OF THE INVENTION

The following description of the background is provided simply as an aid in understanding the present disclosure and is not admitted to describe or constitute prior art to the present disclosure.

Ribonucleic acid protein complexes, i.e., ribonucleoproteins (RNPs), of non-coding ribonucleic acids (ncRNA) play critical roles in numerous biological processes including gene modifications and regulations. Some ncRNAs (guide RNAs) direct RNPs to their targeting genes by base paring. One prominent example of such RNPs is CRISPR-Cas. Variety of CRISPR-Cas such as CRISPR Cas9, base editors and prime editors are in clinical development.

The CRISPR-Cas system is an adaptive immune system of bacteria and composed of clustered regularly interspaced short palindromic DNA repeats and CRISPR-associated genes that protect bacteria against invading phages and mobile genetic elements. CRISPR-Cas9 is being developed for numerous applications in biotechnology and biomedical research and as a gene therapy agent for treatment of multiple conditions including cancers, infectious diseases, and genetic diseases such as sickle cell anemia and Duchenne's muscular dystrophy (DMD), with an increasing number of trials around the world that involve CRISPR in human cells listed in the NIH's database of global clinical trials. Using CRISPR-Cas9 multiplexing gene editing, allogenic universal CAR T cells that are deficient in the TCR beta chain, B2M, PD-1, TCR and CTLA-4 have been produced, with enhanced potency. CRISPR-Cas9 has been applied to silencing/correcting pathogenic proteins in neurodegenerative diseases such as Alzheimer's disease, Huntington's disease, and Parkinson's disease, which should essentially block further progression of symptoms, and it may also be applicable for treatment of dementia with Lewy bodies, frontotemporal dementias, various other tauopathies and amyotrophic lateral sclerosis (ALS). Catalytically impaired Cas9 (dCas9 or nCas9) can target many genomic loci, which has led to technological developments such as base editing, prime editing, epigenetic editing, gene regulation, and chromatin imaging and modeling.

However, to the present, the in vivo gene editing usually gives low to very low editing efficiency which is at least due to the lack of efficient and tissue selective delivery.

Adeno associated viruses (AAVs) are ˜4.7 kb single-stranded DNA viruses that infect humans and primates. AAVs are among the most commonly used vectors for gene delivery, with clinically approved therapeutics such as Luxturna and Zolgensma and a number of clinical trials showing promising results, including for Duchenne Muscular Dystrophy, Lipoprotein Lipase (LPL) deficiency and Hemophilia B among others. Their lack of pathogenicity, superior safety profile, mild immunogenicity, long-term gene expression without genomic integration, and ability to infect both dividing and non-dividing cells, while providing persistent levels of transgene expression makes them favorable systems for in vivo gene transfer. It has been reported AAVs can be used as a viral vector to deliver Cas9 variants of small sizes and Cas12a. Dual AAV vectors were designed to deliver split prime editor. However, because of its limited package size, it is challenging to deliver both Cas9 and transgene or HDR template in a single AAV vector.

AAV has an icosahedral capsid composed of a total sixty copies of three capsid proteins, VP1, VP2, and VP3, which are present in an approximate 1:1:10 ratio. All three capsid proteins share the 533 amino acid C-terminus, while VP1 and VP2 have additional N-terminal extensions. Four basic regions (BRs) were identified and, respectively, named BR1 through BR4. VP1 possesses all four BRs, with BR1, BR2 and BR3 found in the N-terminal domain and BR4 localized to the C-terminal domain. VP2 possesses BR2, BR3, and BR4, and VP3 has BR4 only. These BRs were suggested to play critical roles in capsid's localization into the nucleus (BR1, BR2 and BR3) and capsid assembly (BR4), but are located outside the viral spike region.

AAV serotypes consist of heterogeneous populations with variable VP stoichiometries (It was observed that the majority of the capsids contain between 0-10 copies of VP1, 2-20 copies of VP2, and between 35-55 copies of VP3.), while the presence and abundance of VP1 and VP2 are critical for their roles in endosomal trafficking, endosomal escape, nuclear trafficking, and genome release. Covalent attachment of chemical moieties such as small molecule ligands to the virus capsid can be achieved by amide/urea/thiourea formation via an NHS ester, strained lactam or an isothiocyanate (N═C═S) with exposed amino acid side chains such as lysine or arginine, but such an approach is difficult to control because of the lack of site specificity and gives a heterogeneous mixture of AAV conjugates with varied conjugation sites and stoichiometry. This can decrease the transduction efficiency of the modified viral vector.

This invention teaches virus-RNP conjugates (VRC) for selective delivery of RNP complex into a targeted cell nucleus, and in particular, an AAV-CRISPR RNP conjugate.

Attachment of a guide RNA to the central residues (e.g., 453-457 for AAV2) within the spike of capsid, which are in general more protruding, can result in more efficient conjugation while decrease the perturbations of the biological functions of a viral capsid.

Genetic code expansion (GCE) technology can be used to site-specifically incorporate a noncanonical amino acid (ncAA) with a biorthogonal conjugation handle into capsid proteins, which could be subsequently conjugated with a high degree of selectivity.

In this disclosure, a guide RNA is conjugated at a ncAA site specifically introduced into VP1 or VP2 of AAV capsid. By decoupling the cellular productions of VP1 or VP2 from other viral capsid proteins, this invention teaches guide RNA-AAV capsid conjugates of low stoichiometry (number of copies of VP1 or VP2 or both in the capsid) and thus minimizes unfavorable perturbations by conjugation, which can affect viral assembly, entry, or trafficking.

Alternatively, the C-terminus of AAV capsid protein can be extended and the extension comprises a sortase recognition motif to introduce a conjugation site such as a functional group for click chemistry to link either RNA or protein of an RNP.

RNP assembly of a guide RNA-AAV conjugate and a Cas protein-NLS encoded by a transgene packaged in the AAV vector enables both chemical modifications of guide RNAs critical for efficacy and selectivity, and cell targeting by tissue/cell tropism of AAV for in vivo gene therapy.

In addition, the C-terminus of AAV capsid protein is exposed at the capsid shell, and thus can be extended by a peptide chain to link a protein such as Cas protein or Cas-effector fusion protein to form a protein conjugate, which is capable of cell nucleus-directed gene editing upon binding a guide RNA.

This invention further teaches the uses of VRCs in gene therapy by targeting the guide RNA-AAV conjugate at the AAVS1 site and inserting transgene(s) packaged in the conjugated AAV encoding one or more functional proteins at the same site, wherein the guide RNA is optionally chemically modified to increase its stability and efficacy while decrease its off-target editing.

The adeno-associated virus integration site 1 (AAVS1) locus in intron 1 of PPP1R12C (protein phosphatase 1 regulatory subunit 12C), is known as a genomic “safe harbor” because its disruption does not have adverse effects on the cell, and robust transcription can be used to maintain the expression of an exogenously inserted gene.

Other genomic “safe harbor” sites are known (e.g., CCR5 and hRosa26), and can be applied similarly.

### SUMMARY OF THE INVENTION

This invention pertains to compositions comprising a VRC for selective delivery of the RNP into a targeted cell nucleus. The VRC is a conjugate of an RNA-guided DNA endonuclease such as CRISPR-Cas, the OMEGA system and Fanzors, and other RNA-guided gene-modifying RNPs.

In particular, this invention pertains to compositions comprising a guide RNA-AAV conjugate covalently linked at VP1 or VP2 or both of the capsid and their uses as medicinal agents for treatment of viral infectious diseases and as gene regulation, replacement, disruption and/or correction-based therapeutics.

In one embodiment, the composition further comprises a Cas protein with or without NLS and a transgene or HDR template inserted between the inverted terminal repeats (ITRs) of the AAV genome, of which both Rep and Cap genes are removed.

In one embodiment, the composition further comprises a gene cassette encoding a Cas protein with one or more NLS, and the gene cassette is inserted between two ITR sequences of the AAV genome, of which both Rep and Cap genes are removed.

In another embodiment, the AAV capsid contains one or more guide RNA-VP1 conjugates.

In another embodiment, the AAV capsid contains one or more guide RNA-VP2 conjugates.

In another embodiment, the AAV capsid contains one or more guide RNA-VP1 and guide RNA-VP2 conjugates.

In one embodiment, the guide RNA-AAV conjugate is formed between a guide RNA and AAV capsid by click chemistry (FIG. 4-6). The guide RNA is incorporated with a chemical moiety such as an alkyne, a strained olefin, a tetrazine or an azide, while the capsid protein is modified with a ncAA equipped with the other chemical moiety compatible for click chemistry such an azide, a tetrazine, a strained olefin or an alkyne, respectively (FIG. 5).

In one embodiment, the capsid protein is modified at the C-terminus to add a sortase recognition motif (e.g., LPXTG), which is joined to the C-terminus by a peptide linker (Z). The motif is converted by the sortase enzyme to a chemical moiety compatible for click chemistry such as an alkyne, a strained olefin, a tetrazine or an azide (FIG. 6).

In some embodiments, Z is a peptide of 1-10 amino acids (aa) in length.

In some embodiments, Z is a peptide of 10-20 amino acids (aa) in length.

In some embodiments, Z is a peptide of 20-30 amino acids (aa) in length. In some embodiments, Z is a peptide of 30-50 amino acids (aa) in length.

In some embodiments, the sortase recognition motif is extended (e.g., LPXTGXₙ, wherein n is >1, X and Xₙ can be any amino acid, any two of Xs can be either different or the same.).

In one embodiment, the guide RNA-AAV conjugate is formed between a guide RNA and AAV capsid by dose-controlled conjugation at primary amines of exposed lysine's side chains. The guide RNA is equipped with a phenylisothiocyanate, NHS or a β-lactam anchor.

In one embodiment, the guide RNA-AAV conjugate is further coated with cell-targeting ligands, aptamers, peptides, polysaccharides or PEG to fine-tune AAV tropism and enhance cell targeting in specific tissues, and decrease its interactions with neutralizing antibodies (epitope masking) wherein such coating is dose-controlled and does not impact the trafficking of the modified virus and hence the expression level of its gene products.

In one embodiment, the guide RNA-AAV conjugate targets a genomic “safe harbor” (e.g., AAVS1, CCR5, and hRosa26) and inserts transgene(s) packaged in the conjugated AAV encoding one or more functional proteins wherein the guide RNA comprises one or more non-nucleotide linkers and thus is an LgRNA.

In one embodiment, more than one functional protein is introduced into host cells by multiplexing gene insertions with different transgenes separately packaged in AAV viral vectors of distinct gRNA-AAV conjugates.

In one embodiment, more than one functional protein is produced from a single inserted DNA encoding proteins joined by T2A (Thosea asigna virus 2A) or P2A (porcine teschovirus-1 2A) self-cleaving peptide.

In another embodiment, the guide RNA-AAV conjugate targeting AAVS1 and inserting transgene(s) packaged in the conjugated AAV and encoding one or more functional proteins wherein the guide RNA is chemically modified to increase its stability and efficacy while decrease its off-target editing.

The guide RNA(s) of the AAV conjugates is a chemically modified crRNA (for CRISPR systems with absent tracrRNA), dual guides (crRNA and tracrRNA) an sgRNA or an LgRNA oligonucleotide, comprising nucleotides modified at sugar moieties such as 2′-deoxyribonucleotides, 2′-methoxyribonucleotides, 2′-F-ribonucleotides, 2′-F-arabinonucleotides, 2′-O,4′-C-methylene nucleotides (LNA), unlocked nucleotides (UNA), nucleoside phosphonoacetates (PACE), thiophosphonoacetates (thioPACE), and phosphoromonothioates:

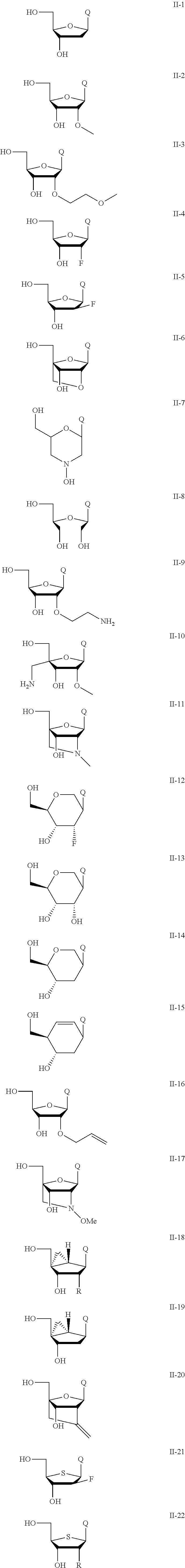

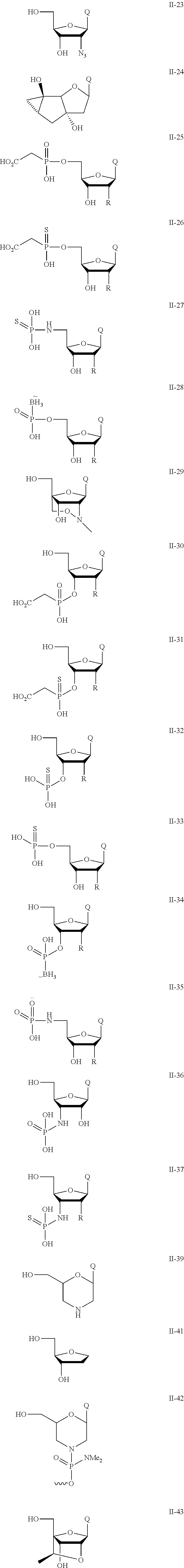

wherein Q is a nucleobase and R is H, OH, F, OMe, or OCH₂CH₂OCH₃; The chemically modified crRNA, dual guides, sgRNA or lgRNA oligonucleotides optionally comprise modified nucleotide base moieties such as G-clamps, A-clamps and other modified bases:

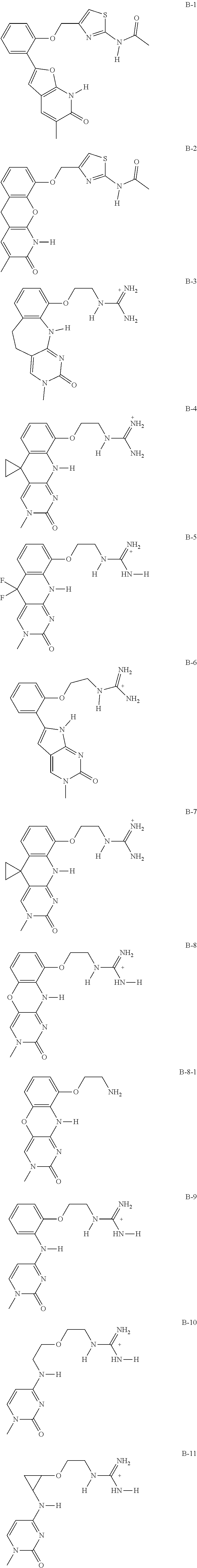

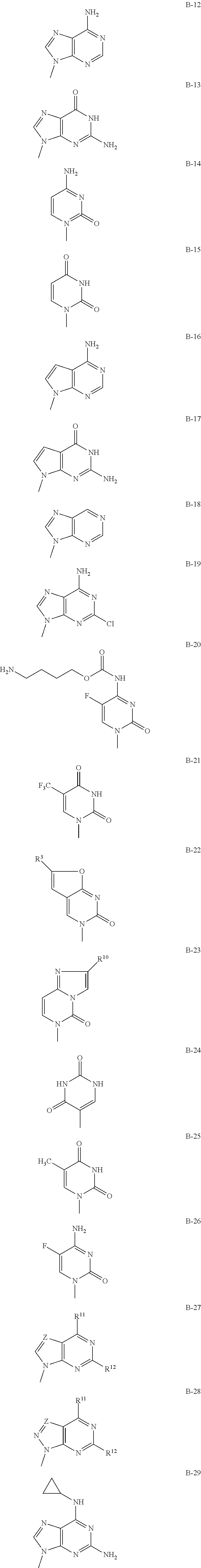

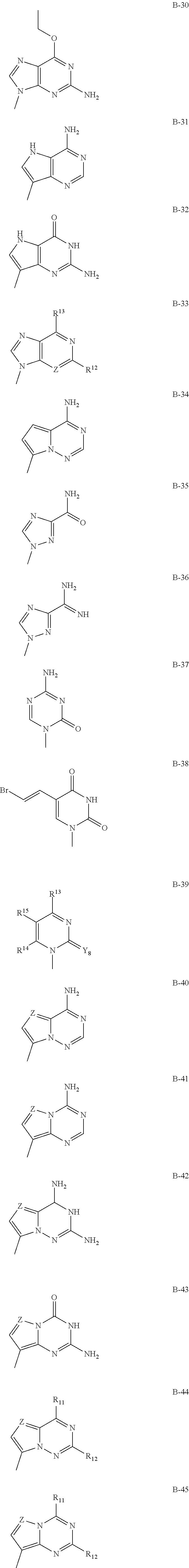

wherein:
(i) Z is N or CR¹⁶; (ii) R⁹, R¹⁰, R¹¹, R¹², R¹³, R¹⁴, R¹⁵ and R¹⁶ are independently H, F, Cl, Br, I, OH, OR′, SH, SR′, SeH, SeR′, NH₂, NHR′, NHOH, NHOR′, NR′OR′, NR′₂, NHNH₂, NR′NH₂, NR′NHR′, NHNR′₂, NR′NR′₂, lower alkyl of C₁-C₆, halogenated (F, Cl, Br, I) lower alkyl of C₁-C₆, lower alkenyl of C₂-C₆, halogenated (F, Cl, Br, I) lower alkenyl of C₂-C₆, CN, lower alkynyl of C₂-C₆, halogenated (F, Cl, Br, I) lower alkynyl of C₂-C₆, lower alkoxy of C₁-C₆, halogenated (F, Cl, Br, I) lower alkoxy of C₁-C₆, CN, CO₂H, CO₂R′, CONH₂, CONHR′, CONR′₂, CH═CHCO₂H, or CH═CHCO₂R′, wherein R′ is an optionally substituted alkyl, which includes, but is not limited to, H, an optionally substituted C₁-C₂₀ alkyl, an optionally substituted lower alkyl, an optionally substituted cycloalkyl, an optionally substituted alkynyl of C₂-C₆, an optionally substituted lower alkenyl of C₂-C₆, an optionally substituted aryl, an optionally substituted heteroaryl, an optionally substituted sulfonyl, or optionally substituted acyl, which includes but is not limited to C(═O) alkyl, or alternatively, in the instance of NR′₂, each R′ comprise at least one C atom that are joined to form a heterocycle comprising at least two carbon atoms.

In some embodiments, chemical modifications of guide RNAs at either sugar or base moiety or both optimize the complementary recognition of the guide-target duplex to improve the cutting efficiency and lower the off-target effects.

In some embodiments, chemical modifications of guide RNAs at either sugar or base moiety or both optimize the shape complementarity between Cas protein and the minor and major grooves of the guide-target duplex to improve the efficiency and lower the off-target effects.

The Cas protein is selected from Cas9 variants comprising SpCas9, St1Cas9, SaCas9, NmCas9, etc. (Jin et al. Adv. Sci. 2020, 1902312; Doudna, J. A. Nature 2020, 578, 229).

The Cas protein can be alternatively any single protein effector of other class 2 CRISPR systems (Type V and VI), such as a Cas12 (a, b, c, e, g, h, i, etc.), Cas13 and Cas14 protein.

In some embodiments, the Cas protein is a fusion protein comprising a Cas protein and an effector protein joined by a peptide linker.

In some embodiments, the Cas protein is a fusion protein comprising a Cas protein and a DNA-directed DNA polymerase joined by a peptide linker.

In some embodiments, the Cas protein is a Cas fusion protein comprising a deactivated Cas protein (dCas) and a nucleotide deaminase joined by a peptide linker.

In some embodiments, the Cas protein is Cas fusion protein comprising a nickase and a reverse transcriptase joined by a peptide linker.

In certain embodiments, the AAV vector can be substituted with engineered retrovirus, lentivirus, HSV, adenovirus, etc. to package genes of sizes larger than AAV package capacity. Examples of such genes include DNAs encoding Cas9 fusion proteins of base editors and prime editors and large genes in gene replacement therapy.

In some embodiments, guide RNA-virus conjugate is covalently linked by a peptide linker joining the viral capsid protein and a Cas protein, i.e., a Cas-capsid fusion protein. The guide RNA conjugates with the virus via its binding to the Cas protein, wherein the Cas protein is optionally fused with another effector protein (e.g., a DNA directed DNA polymerase, a reverse transcriptase, and etc.).

In some embodiments, guide RNA-AAV conjugate is covalently linked by a peptide linker joining the viral capsid protein and a Cas protein, i.e., a Cas-capsid fusion protein. The guide RNA conjugates with the virus via its binding to the Cas protein, wherein the Cas protein is optionally fused with another effector protein (e.g., a DNA directed DNA polymerase, a reverse transcriptase) and the capsid protein is VP1 or VP2.

In some embodiments, the Cas-capsid fusion protein is encoded by a DNA (e.g., a plasmid) and the peptide linker is located between the C-terminus of Capsid protein and the N-terminus of the Cas protein or the Cas fusion protein.

In some embodiments, the Cas-capsid fusion protein is delivered as its mRNA.

The above embodiments for CRISPR-Cas are applicable to other gene modifying RNPs.

### DETAILED DESCRIPTION OF THE INVENTION

The present invention relates to virus-ribonucleic acid protein complex conjugates (VRC), wherein a viral capsid protein is covalently linked to a ribonucleic acid protein complex, i.e., a ribonucleoprotein (RNP), for selective delivery of the RNP into a targeted cell nucleus. These VRCs include conjugates of RNA-guided DNA endonucleases such as CRISPR-Cas, the OMEGA system and Fanzors, and other RNA-guided gene-modifying RNPs.

In particular, the invention relates to compositions of a guide RNA-AAV conjugate for cell-targeted precise CRISPR gene editing.

The most commonly used type of CRISPR system for gene regulation, disruption or correction to date is type II represented predominantly by Cas9. CRISPR-Cas9 is a naturally occurring defense system of bacteria. The CRISPR Cas9 endonuclease is activated by the binding of crRNA: tracrRNA and responds specifically to the DNA sequence (target strand) complementary to the spacer in crRNA and cleaves it upon the recognition of a protospacer adjacent motif (PAM) on the 3′-end of the non-target DNA strand. The presence of tracrRNA and Cas9 is required for processing pre-crRNA into individual crRNA by a double-stranded RNA specific ribonuclease, RNase III, forming crRNA: tracrRNA duplexes. This duplex was fused into an artificial single guide RNA (sgRNA via nucleotide tetraloop or lgRNA via a non-Nucleotide linker) for genome engineering purpose and other applications.

Attractive alternatives include other class 2 CRISPR systems such as Type V and VI.

This invention pertains a synthetic conjugate for delivery of CRISPR gene editing components into a cell nucleus. The synthetic conjugate comprises a guide RNA and a capsid protein covalently linked. The covalent linker is either intact or cleaved after the conjugate is localized to a targeted nucleus.

In some embodiments, the guide RNA is bound by a Cas protein in a RNP complex. A transgene or an HDR template is packaged in the viral vector comprising at least one copy of a guide RNA-capsid protein conjugate. Therefore, the delivery system comprises both a CRISPR RNP complex and a DNA template for precise gene editing or insertion.

In some embodiments, the protein effector bound by the guide RNA is a fusion protein comprising a Cas protein and a DNA directed DNA polymerase, wherein the Cas protein is capable of cleaving the target gene to form a double strand break at the target site. The capsid protein is integrated into an engineered AAV vector, which is packaged with a transgene or a HDR template.

In some embodiments, the protein effector bound by the guide RNA is a fusion protein comprising a Cas protein and a DNA directed DNA polymerase, wherein the Cas protein is a nickase capable of cleaving the target gene to form a single strand break at the target site.

In some embodiments, the protein effector bound by the guide RNA is a fusion protein comprising a Cas protein and an integrase.

In some embodiments, the bound Cas protein has no added NLS in its peptide sequence.

In other embodiments, a RNP complex in a target nucleus is formed from a capsid-conjugated guide RNA and a Cas protein encoded by a transgene packaged in a viral vector. The viral vector comprises at least one copy of the guide RNA-capsid protein conjugate. Therefore, the delivery system comprises a conjugated CRISPR guide RNA and a conjugated viral vector with its Rep and Cap genes replaced by a DNA encoding a Cas protein.

In other embodiments, a RNP complex in a target nucleus is formed from a capsid-conjugated guide RNA and a Cas protein encoded by a transgene packaged in a viral vector. The viral vector comprises at least one copy of the guide RNA-capsid protein conjugate. Therefore, the delivery system comprises a conjugated CRISPR guide RNA and a conjugated viral vector with its Rep and Cap genes replaced by a DNA encoding a Cas fusion protein comprising a deactivated Cas protein (dCas) and a nucleotide deaminase.

In some embodiments, guide RNA is a prime editing guide RNA (pegRNA/epegRNA), and the conjugated viral vector encodes a Cas fusion protein comprising a nickase and a reverse transcriptase (i.e., an RNA-directed DNA polymerase).

In some embodiments, guide RNA is a chemically ligated prime editing guide RNA (lg-pegRNA/lg-epegRNA) comprising at least one internal non-nucleotide linker, and the conjugated viral vector encodes a Cas fusion protein comprising a nickase and a reverse transcriptase.

In some embodiments, guide RNA is covalently linked to a DNA template, and thus is a Seek-Tag-Amend-Release (STAR) editing guide RNA (Zhong, PCT/US2020/036860, filed on Jun. 10, 2020, the entire said invention is incorporated herein by reference), and the conjugated viral vector encodes a Cas protein.

In some embodiments, guide RNA is covalently linked to a DNA template, and thus is a Seek-Tag-Amend-Release (STAR) editing guide RNA (Zhong, PCT/US2020/036860, filed on Jun. 10, 2020, the entire said invention is incorporated herein by reference), and the conjugated viral vector encodes a Cas fusion protein comprising a nickase and a DNA-directed DNA polymerase.

In some embodiments, the capsid of the viral vector is further covalently coated with cell-targeting ligands, aptamers or peptides or epitope masking molecules such as PEG.

In some embodiments, the conjugated viral vector is an engineered AAV.

In some embodiments, this invention pertains to compositions of a guide RNA-AAV conjugate covalently linked at VP1 or VP2 or both of the capsid and their uses as medicinal agents for treatment of viral infectious diseases and as gene regulation, disruption and/or correction-based therapeutics.

In some embodiments, the guide RNA-AAV conjugate targets AAVS1 to form a double strand break, and cellular enzymes insert transgene(s) packaged in the conjugated AAV encoding one or more functional proteins wherein the guide RNA is bound to a Cas protein and is chemically modified to increase its stability and efficacy while decrease its off-target editing.

In some embodiments, the guide RNA-AAV conjugate targeting AAVS1 is modified by substitution of one or more cytosines in its spacer with G-clamp nucleotides.

In some embodiments, the guide RNA of the guide RNA-viral capsid conjugate targets AAVS1 to form a double strand break, and cellular enzymes insert transgene(s) packaged in the conjugated viral vector encoding one or more functional proteins, wherein the guide RNA is bound to a Cas protein and is chemically modified to increase its stability and efficacy while decrease its off-target editing; and the viral vector has a package capacity larger than that of AAV. Examples of such viral vectors include retrovirus, lentivirus, HSV, and adenovirus, and these viral vectors are incorporated with one or more guide RNA-viral capsid conjugates.

In some embodiments, the guide RNA of the guide RNA-viral capsid conjugate targets AAVS1 to form a single strand break, and cellular enzymes insert transgene(s) packaged in the conjugated viral vector encoding one or more functional proteins, wherein the guide RNA is bound to a Cas protein and is chemically modified to increase its stability and efficacy while decrease its off-target editing; and the viral vector has a package capacity larger than that of AAV. Examples of such viral vectors include retrovirus, lentivirus, HSV, and adenovirus, and these viral vectors are incorporated with one or more guide RNA-viral capsid conjugates.

In some embodiments, the guide RNA of the guide RNA-viral capsid conjugate targets AAVS1 to form a double strand break, and a Cas protein-fused DNA-directed polymerase inserts transgene(s) packaged in the conjugated viral vector encoding one or more functional proteins, wherein the guide RNA is bound to a Cas protein and is chemically modified to increase its stability and efficacy while decrease its off-target editing; and the viral vector has a package capacity larger than that of AAV. Examples of such viral vectors include retrovirus, lentivirus, HSV, and adenovirus, and these viral vectors are incorporated with one or more guide RNA-viral capsid conjugates.

In some embodiments, the guide RNA of the guide RNA-viral capsid conjugate targets AAVS1 to form a single strand break, and a Cas protein-fused DNA-directed polymerase inserts transgene(s) packaged in the conjugated viral vector encoding one or more functional proteins, wherein the guide RNA is bound to a Cas protein and is chemically modified to increase its stability and efficacy while decrease its off-target editing; and the viral vector has a package capacity larger than that of AAV. Examples of such viral vectors include retrovirus, lentivirus, HSV, and adenovirus, and these viral vectors are incorporated with one or more guide RNA-viral capsid conjugates.

In some embodiments, the guide RNA-virus conjugate targeting AAVS1 is modified by substitution of one or more cytosines in its spacer with G-clamp nucleotides.

Other genomic “safe harbor” sites are known (e.g., CCR5 and hRosa26), and can be applied similarly.

The above embodiments for CRISPR-Cas are applicable to other RNPs.

### BRIEF DESCRIPTION OF THE DRAWINGS

FIG. 1 shows: schematic structure of a VRC comprising a gRNA-AAV-ligand conjugate and its components (AAV-CRISPR-Cas9 VAC as an example).

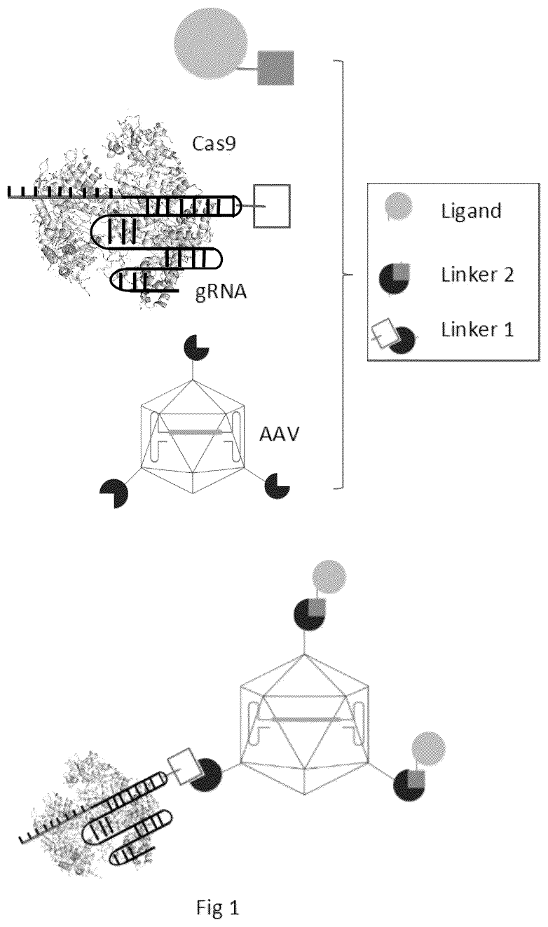

FIG. 2 shows: the components (CRISPR RNP complex and a transgene) in cell nucleus after transductions of gRNA-AAV-ligand conjugates (in a VRC). The RNP complex is either free of capsid protein (2-A) or with a VP1 or VP2 covalently attached (2-B). The transgene encodes one or more functional proteins, or a Cas protein/Cas fusion protein (with a virus of an appropriate packaging capacity).

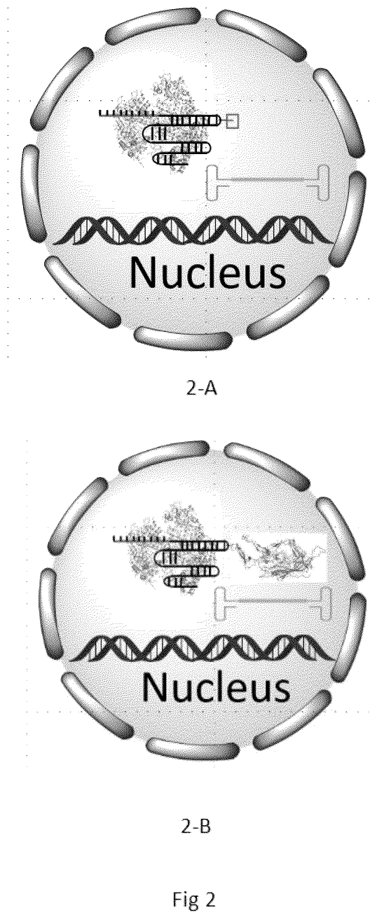

FIG. 3 shows: the spike of AAV-DJ for incorporating a ncAA.

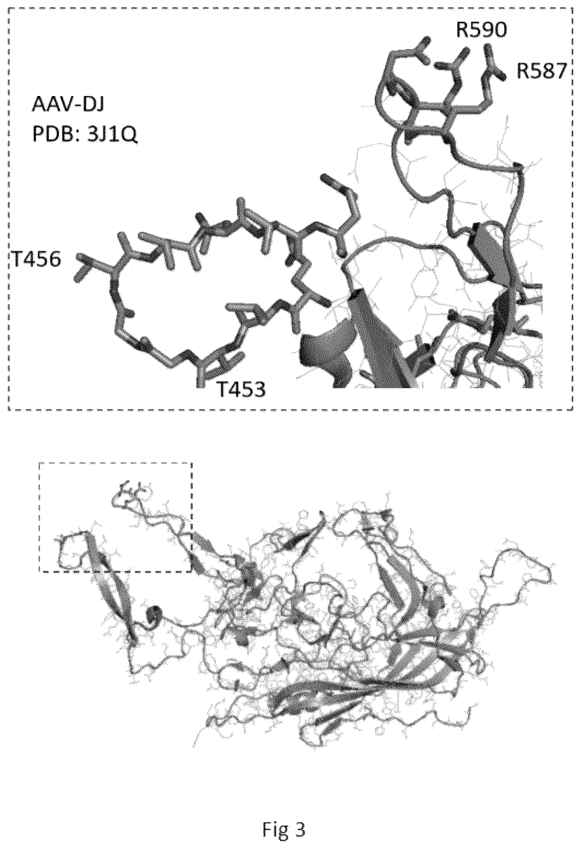

FIG. 4 shows: Schematic structure of an example of 3′-azido modified gRNA (CRISPR-Cas9).

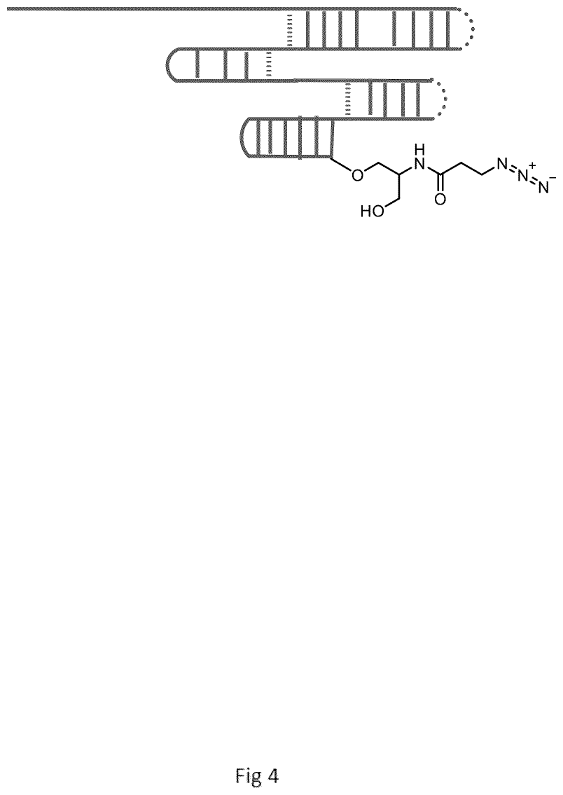

FIG. 5 shows: Schematic structures of gRNA-VP1 or VP2 conjugates with an example linker. Because the spike is common for all three capsid proteins (VP1, VP2, and VP3), this structure moiety is the same when the conjugating site is located in VP3. Examples are given: gRNA-AAV conjugates of LgRNA/sgRNA (5-A, loops can either by a nucleotide linker or a non-nucleotide linker), dual guides (5-B, crRNA and tracrRNA) or crRNA (5-C, for CRISPR-Cas systems without a traceRNA).

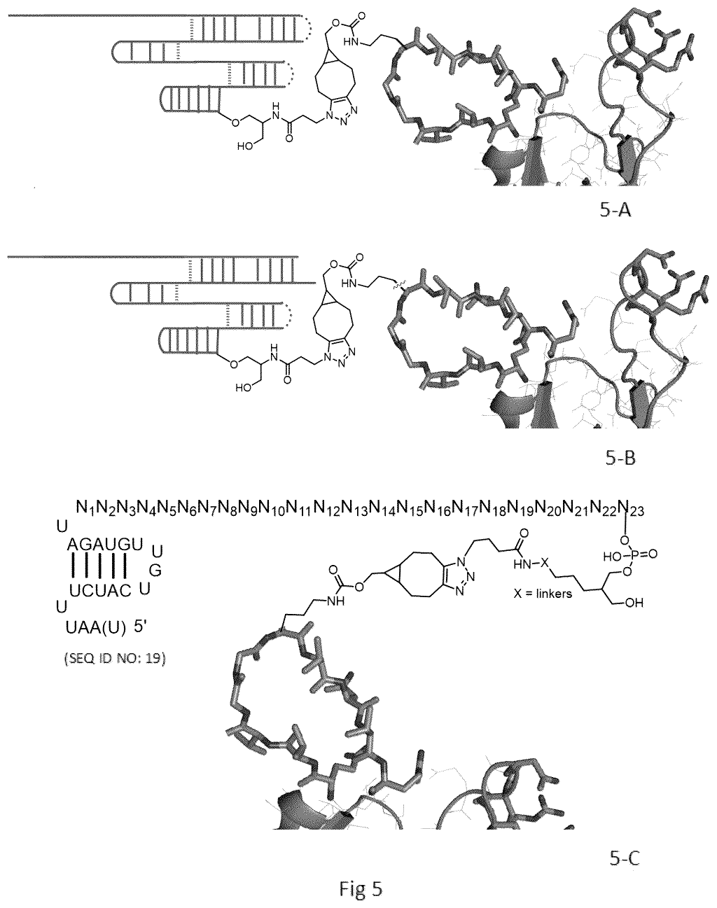

In a dual guides-AAV conjugate (5-B), the conjugating sites of guide RNA is at 5′-/3′-end of crRNA or tracrRNA, at the nNt-Linker or at a nucleotide unbound by Cas protein.

CrRNA-VP1/VP2 conjugate (5-C) is linked at 5′-/3′-end of the guide RNA.

FIG. 6 shows: a method to produce a guide RNA-AAV conjugate linked at the C terminus of VP1 or VP2, comprising:

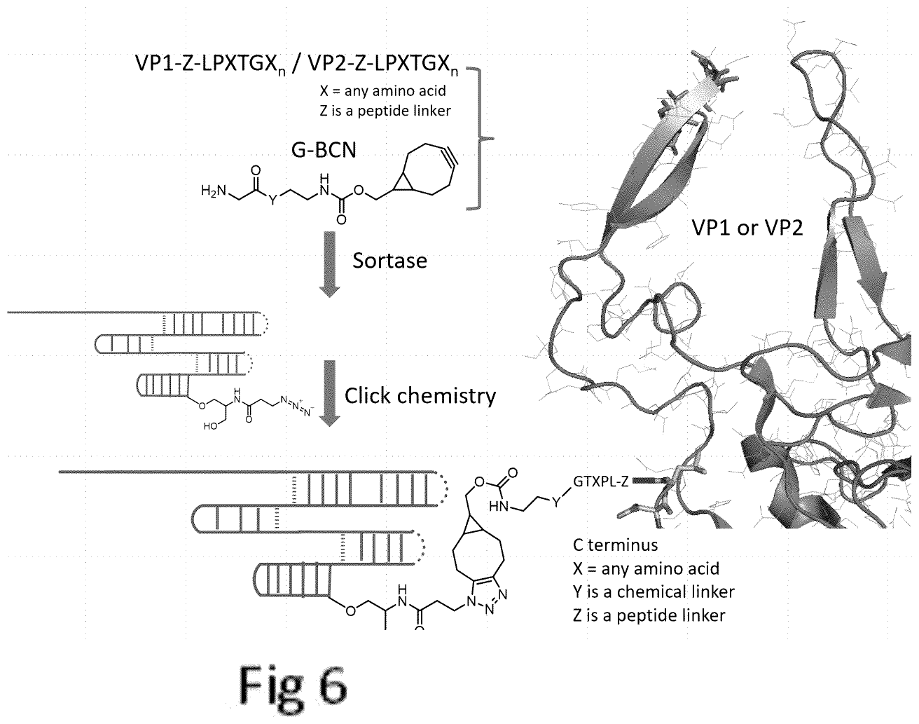

Step 1. The C terminus of VP1 or VP2 is modified to add a LPXTG motif linked via a peptide linker (Z), wherein X and Xn can be any amino acid, any two of Xs can be either different or the same, and the terminal Xn is optional;

Step 2. The VP1 or VP2 is converted to a clickable derivative (e.g., BCN) by a sortase; and

Step 3. The VP1 or VP2 is conjugated with a guide RNA via a click reaction.

### DEFINITION

The definitions of terms used herein are consistent to those known to those of ordinary skill in the art, and in case of any differences the definitions are used as specified herein instead.

The term of “virus-ribonucleic acid protein complex conjugates” (VRC) as used herein refers to a conjugate for selective delivery of the RNP into a cell nucleus. A VRC comprises a virus and a ribonucleic acid protein complex (RNP). The two components are linked by a covalent linker or linked by non-covalent binding between the components' modifiers (e.g., streptavidin/biotin, an aptamer/a binding partner and a peptide tag/a protein binding partner). The covalent linker joins either the RNA or the protein of the RNP to a viral capsid protein.

The term of “ribonucleoprotein” (RNP) is a complex of ribonucleic acid and RNA-binding protein. Examples include RNA-guided DNA endonucleases such as CRISPR-Cas, the OMEGA system and Fanzors.

The term “nucleoside” as used herein refers to a molecule composed of a heterocyclic nitrogenous base, containing an N-glycosidic linkage with a sugar, particularly a pentose. An extended term of “nucleoside” as used herein also refers to acyclic nucleosides and carbocyclic nucleosides.

The term “nucleotide” as used herein refers to a molecule composed of a nucleoside monophosphate, di-, or triphosphate containing a phosphate ester at 5′-, 3′-position or both. The phosphate can also be a phosphonate, phosphoramidate, phosphorodiamidate, phosphonoacetate (PACE), thiophosphonoacetate (thioPACE), or phosphoromonothioate.

The term of “oligonucleotide” (ON) is herein used interchangeably with “polynucleotide”, “nucleotide sequence”, and “nucleic acid”, and refers to a polymeric form of nucleotides of any length, either deoxyribonucleotides or ribonucleotides, or analogs thereof. An oligonucleotide may comprise one or more modified nucleotides, which may be imparted before or after assembly of such an oligonucleotide. The sequence of nucleotides may be interrupted by non-nucleotide components.

The term of “CRISPR-Cas system” refers a prokaryotic immune system that confers resistance to foreign genetic elements such as those present within plasmids and phages that provides a form of acquired immunity. RNA harboring the spacer sequence helps Cas (CRISPR-associated) proteins recognize and cut foreign pathogenic DNA. Other RNA-guided Cas proteins cut foreign RNA. CRISPR are found in approximately 50% of sequenced bacterial genomes and nearly 90% of sequenced archaea. The system is being engineered for gene regulation and editing, insertion, disruption and/or correction in eukaryotic cells.

The term of “CRISPR/Cas9” refers to the type II CRISPR-Cas system such as SpCas9 from Streptococcus pyogenes. The type II CRISPR-Cas system comprises protein Cas9 and two noncoding RNAs (crRNA and tracrRNA). These two noncoding RNAs were further fused into one single guide RNA via a tetraloop (sgRNA) and a chemically ligated guide RNA via one or more nNt-Linkers (lgRNA). The Cas9/sgRNA or Cas9/1gRNA complex binds double-stranded DNA sequences that contain a sequence match to the first 17-20 nucleotides of the guide RNA(s) and immediately before a protospacer adjacent motif (PAM). Once bound, two independent nuclease domains (HNH and RuvC) in Cas9 each cleaves one of the DNA strands 3 bases (HNH) or more (RuvC) upstream of the PAM, leaving a DNA double stranded break (DSB).

The term of “Cas protein” refers to a class 2 CRISPR-Cas protein.

The term of “off-target effects” refers to non-targeted cleavage of the genomic DNA target sequence by Cas9 or any other Cas protein despite imperfect matches between the gRNA sequence and the genomic DNA target sequence. Single mismatches of the gRNA can be permissive for off-target cleavage by Cas9. Off-target effects were reported for all the following cases: (a) same length but with 1-5 base mismatches; (b) off-target site in target genomic DNA has one or more bases missing (‘deletions’); (c) off-target site in target genomic DNA has one or more extra bases (‘insertions’).

The term of “guide RNA” (gRNA) refers to the RNA component of an RNP, such as a CRISPR-Cas system, an RNA-guided DNA endonuclease and an RNP gene modifier. The guide RNA is bound by the RNP protein and directs the RNP to the target gene by base pairing. The guide RNA comprises optional non-nucleotide linkers, and is ether a single-RNA molecule or a chemically ligated RNA (lgRNA).

The term of “guide RNA” (gRNA) of a CRISPR-Cas system refers to the RNA component, e.g. crRNA, dual guide RNAs, a synthetic fusion of crRNA and tracrRNA via a tetraloop (GAAA) (defined as sgRNA) or other chemical linkers such as an nNt-Linker (defined as lgRNA), which is used interchangeably with “chimeric RNA”, “chimeric guide RNA”, “single guide RNA” and “synthetic guide RNA”. The gRNA of CRISPR-Cas9 contains secondary structures of the repeat: anti-repeat duplex, stem loops 1-3, and the linker between stem loops 1 and 2.

The term of “dual RNAs” or “dual guide RNAs” refers to a hybridized complex of the short CRISPR RNAs (crRNA) and the trans-activating crRNA (tracrRNA). The crRNA hybridizes with the tracrRNA to form a crRNA: tracrRNA duplex, which is loaded onto a Cas protein to direct the cleavage of cognate DNA sequences bearing appropriate protospacer-adjacent motifs (PAM).

The term of “lgRNA” refers to a guide RNA (gRNA) joined by chemical ligations to form non-nucleotide linkers (nNt-linkers) between a crgRNA and a tracrgRNA, or at other sites.

The terms of “dual lgRNA”, “triple lgRNA” and “multiple lgRNA” refer to hybridized complexes of the synthetic guide RNA fused by chemical ligations via non-nucleotide linkers. A dual tracrgRNA is formed by chemical ligation between a tracrgRNA1 and a tracrgRNA2 (RNA segments of ˜30 nt), and a crgRNA (˜30 nt) is fused with a dual tracrgRNA to form a triple lgRNA duplex, which is loaded onto Cas9 to direct the cleavage of cognate DNA sequences bearing appropriate protospacer-adjacent motifs (PAM). Each RNA segment can be readily accessible by chemical manufacturing and compatible to extensive chemical modifications.

The term “guide sequence” refers to the about 20 bp sequence within the guide RNA that specifies the target site and is herein used interchangeably with the terms “guide” or “spacer”. The term “tracr mate sequence” may also be used interchangeably with the term “direct repeat(s)”.

The term of “crgRNA” refers to a crRNA equipped with chemical functions for conjugation/ligation. The oligonucleotide may be chemically modified close to its 3′-end, any one or several nucleotides, or for its full sequence. A crgRNA may also be prepared by in vitro transcription at the presence of a RNA polymerase such as bacteriophage T7 RNA polymerase, and the conjugating chemical function, e.g., amine and alkyne, is incorporated at its 5′-end (preferably as 5′-GU . . . or 5′-GC . . . primers with modifications), and 3′-end from a nucleoside triphosphate analogue, e.g. CTP and UTP:

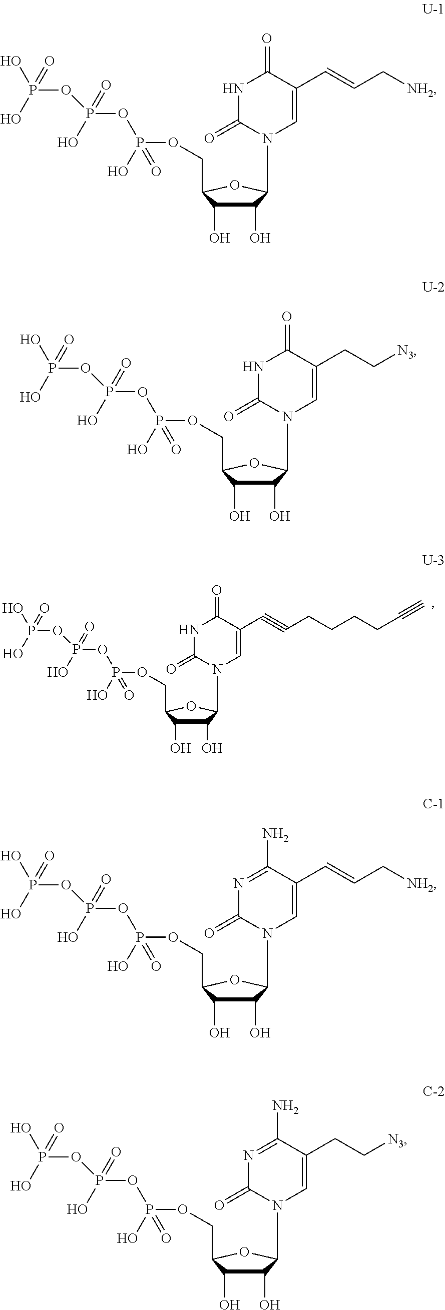

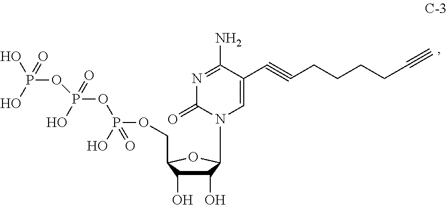

and etc.

The term of “tracrgRNA” refers to a tracrRNA equipped with chemical functions for conjugation/ligation. The oligonucleotide may be chemically modified at any one or several nucleotides, or for its full sequence by chemical synthesis. A tracrgRNA may also be prepared by in vitro transcription at the presence of a RNA polymerase such as bacteriophage T7 RNA polymerase, and the conjugating chemical function, e.g., amine and alkyne, is incorporated at its 5′-end (preferably as 5′-GU . . . or 5′-GC . . . primers with modifications), and 3′-end from a nucleoside triphosphate analogue, e.g. CTP and UTP.

The term of “the protospacer adjacent motif (PAM)” refers to a DNA sequence immediately following the DNA sequence targeted by Cas9 in the CRISPR bacterial adaptive immune system, including NGG, NNNNGATT, NNAGAA, NAAAC, and others from different bacterial species where N is any nucleotide. In CRISPR-Cas12a system, “PAM” refers to a DNA sequence such as TTTN immediately before the targeted DNA sequence.

The term of “chemical ligation” refers to joining together synthetic oligonucleotides via an nNt-linker by chemical methods such as click ligation (the azide-alkyne reaction to produce a triazole linkage), thiol-maleimide reaction, and formations of other chemical groups.

The term of “complementary” refers to the ability of a nucleic acid to form hydrogen bond(s) with another nucleic acid sequence by either traditional Watson-Crick or other non-traditional types. Cas9 contains two nuclease domains, HNH and RuvC, which cleave the DNA strands that are complementary and non-complementary to the 20 nucleotide (nt) guide sequence in crRNAs, respectively.

The term of “a donor template” refers to a transgene cassette or a gene-editing-sequence flanked with homologous regions to recombine with the host loci and replace the mutated DNA with the correct sequence by HDR/SSTR. A donor template can be an ssDNA or a dsDNA or a plasmid/vector, and may be chemically conjugated to guide RNA(s) or Cas protein via a covalent linker.

The term of “gene editing sequence”, “gene-editing-sequence” or “gene_editing_sequence” refers to the sequence contained in a donor template sequence to introduce expected gene editing, between the two homology arms identical to the DNA fragments flanking the cleavage site.

The term of “Hybridization” refers to a reaction in which one or more polynucleotides form a complex that is stabilized via hydrogen bonding between the bases of the nucleotide residues. The complex may comprise two strands forming a duplex structure, three or more strands forming a multi stranded complex, a single self-hybridizing strand, or any combination of these. A sequence capable of hybridizing with a given sequence is referred to as the “complement” of the given sequence.

The term of “noncanonical amino acid (ncAA)” refers to a non-natural amino acid, which can be incorporated into proteins via genetic code expansion (GCE). The archaea-derived pyrrolysyl-tRNA synthetase (PylRS)/tRNAPyl pair has been used to incorporate various ncAAs, including BCNK, in response to the repurposed natural STOP codon. The incorporation of BCNK was shown to have a minimal impact on AAV transduction efficiency and enable chemoselective labeling of the capsid using strain-promoted azide-alkyne click chemistry (SPAAC).

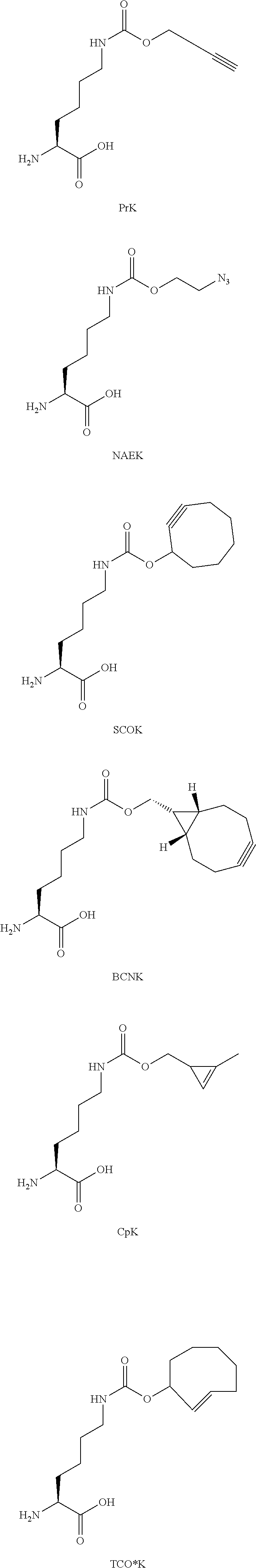

The synonymous terms “hydroxyl protecting group” and “alcohol-protecting group” as used herein refer to substituents attached to the oxygen of an alcohol group commonly employed to block or protect the alcohol functionality while reacting other functional groups on the compound. Examples of such alcohol-protecting groups include but are not limited to the 2-tetrahydropyranyl group, 2-(bisacetoxyethoxy) methyl group, trityl group, trichloroacetyl group, carbonate-type blocking groups such as benzyloxycarbonyl, trialkylsilyl groups, examples of such being trimethylsilyl, tert-butyldimethylsilyl, tert-butyldiphenylsilyl, phenyldimethylsilyl, triiospropylsilyl and thexyldimethylsilyl, ester groups, examples of such being formyl, (C1-C10) alkanoyl optionally mono-, di- or tri-substituted with (C1-C6) alkyl, (C1-C6) alkoxy, halo, aryl, aryloxy or haloaryloxy, the aroyl group including optionally mono-, di- or tri-substituted on the ring carbons with halo, (C1-C6) alkyl, (C1-C6) alkoxy wherein aryl is phenyl, 2-furyl, carbonates, sulfonates, and ethers such as benzyl, p-methoxybenzyl, methoxymethyl, 2-ethoxyethyl group, etc. The choice of alcohol-protecting group employed is not critical so long as the derivatized alcohol group is stable to the conditions of subsequent reaction(s) on other positions of the compound of the formula and can be removed at the desired point without disrupting the remainder of the molecule. Further examples of groups referred to by the above terms are described by J. W. Barton, “Protective Groups In Organic Chemistry”, J. G. W. McOmie, Ed., Plenum Press, New York, N.Y., 1973, and G.M. Wuts, T.W. Greene, “Protective Groups in Organic Synthesis”, John Wiley & Sons Inc., Hoboken, New Jersey, 2007, which are hereby incorporated by reference. The related terms “protected hydroxyl” or “protected alcohol” define a hydroxyl group substituted with a hydroxyl protecting group as discussed above.

The term “nitrogen protecting group,” as used herein, refers to groups known in the art that are readily introduced on to and removed from a nitrogen atom. Examples of nitrogen protecting groups include but are not limited to acetyl (Ac), trifluoroacetyl, Boc, Cbz, benzoyl (Bz), N,N-dimethylformamidine (DMF), trityl, and benzyl (Bn). See also G. M. Wuts, T. W. Greene, “Protective Groups in Organic Synthesis”, John Wiley & Sons Inc., Hoboken, New Jersey, 2007, and related publications.

The term “conjugation”, as used herein, refers to a method for covalently crosslinking drug molecules, proteins or nucleic acids to other molecules using crosslinking reagents. The product of conjugation is referred as “conjugate(s)”. Traditional pharmaceuticals can be linked to monoclonal antibodies to deliver targeted doses, prevent breakdown, decrease immunogenicity, and increase bioavailability in circulation. Nevertheless, CRISPR RNP and viral capsid can alternatively be chemically modified by linking them to other molecules and to form a VRC by joining either covalently or non-covalently via the added moieties.

The term “conjugating site”, as used herein, refers to a chemical moiety which is directly linked to other molecules by conjugation, and a conjugating site can be an amino acid residue, N-terminus or C-terminus of proteins, a nucleoside, a nucleotide, or a phosphate.

The term “PEG,” or “macrogol”, as used herein, refers to polyethylene glycol chains, linear, branched, substituted or unsubstituted. A derivatized linear single PEG chain comprises at least 2 PEG subunits.

The term “PEGylation”, as used herein, refers to the process of both covalent and non-covalent attachment or amalgamation of polyethylene glycol (PEG) polymer chains to molecules and macrostructures, such as a drug, a CRISPR RNP complex, a therapeutic protein or vesicle, which is then described as PEGylated. PEGylation is routinely achieved by incubation of a reactive derivative of PEG with the target molecule.

The term “glycan”, as used herein, refers to polysaccharides or the carbohydrate portion of a glycoconjugate, such as a glycoprotein, glycolipid, or a proteoglycan, even if the carbohydrate is only an oligosaccharide.

The term “polysaccharides”, as used herein, refers compounds consisting of a large number of monosaccharides linked glycosidically.

The term “epitope” or “antigenic determinant”, as used herein, refers to the part of an antigen that is recognized by the immune system, specifically by antibodies, B cells, or T cells. An epitope can be either conformational or linear.

The term “epitope masking”, as used herein, refers to identifying potentially immunogenic peptide sequences and modifying or removing them to prevent detection by the immune system while still maintaining the therapeutic function of the original protein.

The term of “Isotopically enriched” refers to a compound containing at least one atom having an isotopic composition other than the natural isotopic composition of that atom. The term of “Isotopic composition” refers to the amount of each isotope present for a given atom, and “natural isotopic composition” refers to the naturally occurring isotopic composition or abundance for a given atom. As used herein, an isotopically enriched compound optionally contains deuterium, carbon-13, nitrogen-15, and/or oxygen-18 at amounts other than their natural isotopic compositions. Conjugates of CRISPR RNP complexes are optionally isotopically enriched at selected positions to optimize their drug properties based on isotope effects.

As used herein, the terms “therapeutic agent” and “therapeutic agents” refer to any agent(s) which can be used in the treatment or prevention of a disorder or one or more symptoms thereof. In certain embodiments, the term “therapeutic agent” includes a compound provided herein. In certain embodiments, a therapeutic agent is an agent known to be useful for, or which has been or is currently being used for the treatment or prevention of a disorder or one or more symptoms thereof.

The term of “gene therapy” refers to altering a disease-causing gene in a patient or introducing a healthy copy of a mutated gene to a patient to treat genetic diseases. CRISPR/Cas can potentially be used to introduce site specific gene editing to correct disease-causing mutations, or to deliver a correct gene into human genome to fix a defect gene or a desired gene. CRISPR/Cas can potentially be used to remove and/or deactivate episomal HBV cccDNA and integrated viral genomes such as HIV proviral DNA and integrated HBV DNA to cure these infectious diseases.

It is noted that as used, the singular forms “a,” “an,” and “the” include plural referents unless the context clearly dictates otherwise. Thus, for example, reference to “a guide RNA-AAV conjugate” includes a plurality of such complexes. Reference to “the conjugate” includes reference to one or more conjugates and equivalents thereof known to those skilled in the art, and so forth. It is further noted that the claims may be drafted to exclude any optional element. As such, this statement is intended to serve as antecedent basis for use of such exclusive terminology as “solely”, “only” and the like in connection with the recitation of claim elements, or use of a “negative” limitation.

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

### Nucleotides

The guide RNA binds both the protein of RNP and the target DNA.

The guide RNA is either a single-RNA molecule or a chemically ligated RNA comprising one or more internal non-nucleotide linkers.

The guide RNA is optionally chemically modified.

In a CRISPR-Cas system, the guide RNA is a chemically modified crRNA (for CRISPR systems with absent tracrRNA), dual guides (crRNA and tracrRNA), an sgRNA or an LgRNA oligonucleotide, as described below.

In some embodiments, the crRNA and tracrRNA are truncated at 3′-end and 5′-end, respectively:

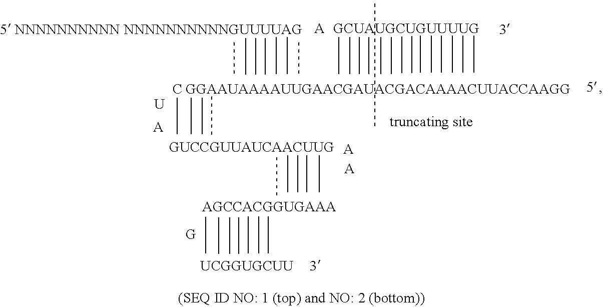

and the duplex ends are rejoined by a small molecule non-nucleotide linker (nNt-linker, ligation1) to form a dual lgRNA:

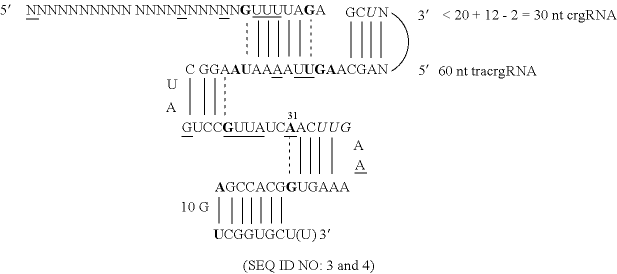

wherein “NNNNNNNNNN NNNNNNNNNN” is a guide sequence of 17-20 nt, and N is preferably a ribonucleotide with intact 2′-OH, and wherein “ [symbol] ” is a chemical nNt-linker.

In some embodiments, the duplex ends are rejoined by a tetraloop (e.g. GAAA) to form an sgRNA:

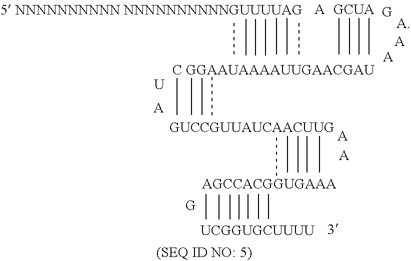

In other embodiments, tracrRNA is a ligated dual oligonucleotide (via ligation2, the inner ligation between tracrgRNA1 and tracrgRNA2), or a multiple oligonucleotide. Non-limiting examples include:

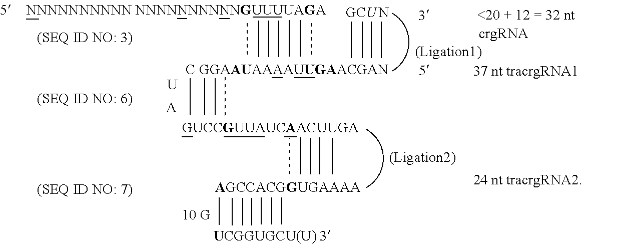

In some embodiments, the crRNA and tracrRNA are further truncated and the ligation site can be located at other positions, as illustrated by non-limiting examples of resulting lgRNAs in U.S. Pat. No. 10,059,940.

In some embodiments, crRNA and tracrRNA are truncated at 3′-end and 5′-end, respectively, and the duplex ends are rejoined by a nucleotide linker such as an aptamer and thus to provide an extended single gRNA with small molecule and protein recognition module(s):

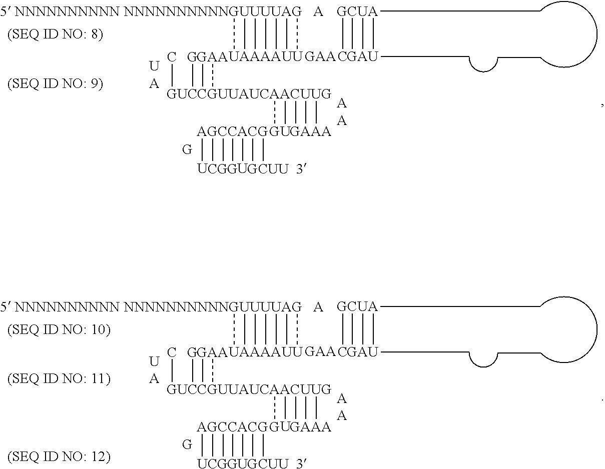

In another embodiment, the crRNA and tracrRNA are truncated at 3′-end and 5′-end, respectively, and the duplex ends are rejoined by a non-nucleotide linker-aptamer conjugate to provide an extended single lgRNA with small molecule and protein recognition module(s):

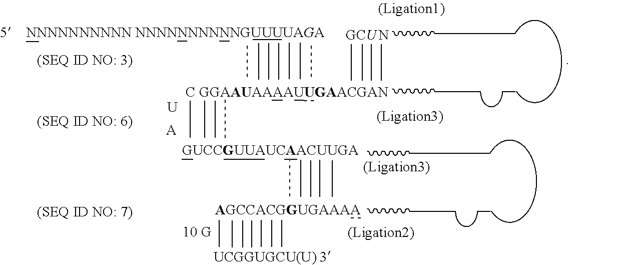

In another embodiment, the stem loop of tracrRNA is split at the GAAA tetraloop, and the duplex ends are rejoined by a non-nucleotide linker-aptamer conjugate to provide an extended tracrRNA with small molecule and protein recognition module(s):

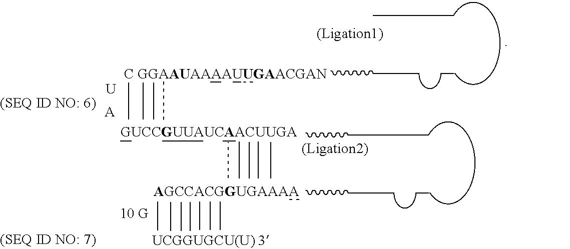

In yet another embodiment, lgRNA is conjugated with an aptamer by a non-nucleotide linker at either of the two GAAA tetraloops or both, or 5′/3′-end of sgRNA, to bind a small molecule or a biopolymer such as a protein or a nucleic acid:

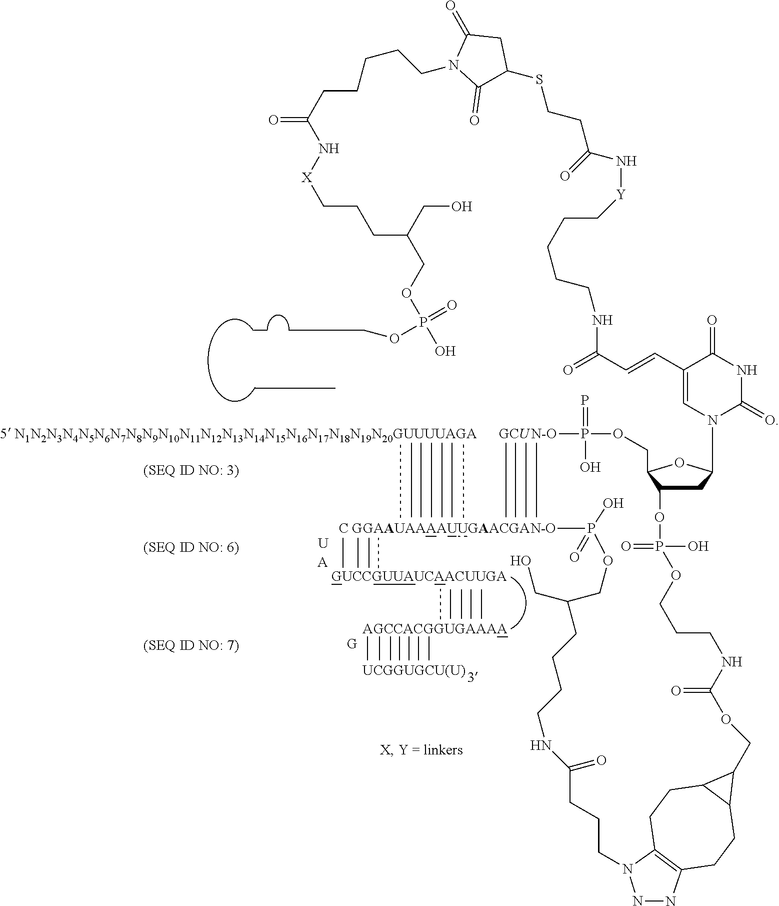

In some embodiments, the crRNA and tracrRNA are shortened by truncation at 3′-end and 5′-end, respectively, and the repeat/anti-repeat duplex comprises a bulge and >12 Watson-Crick base pairs:

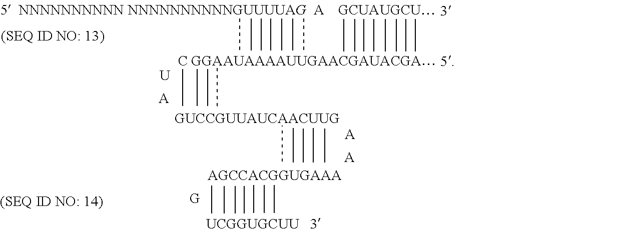

In some embodiments, the crRNA and tracrRNA are joined at 3′-end of tracrRNA and 5′-end of crRNA by a nucleotide linker or a non-nucleotide linker to form a sgRNA or lgRNA, respectively; and the tracrRNA is optionally a ligated tracrRNA comprising one or more than one non-nucleotide linker:

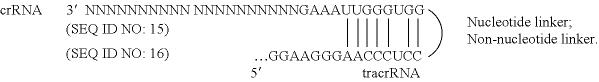

In some embodiments, the guide RNA is covalently linked to a DNA template, and thus is a Seek-Tag-Amend-Release (STAR) editing guide RNA (Zhong, PCT/US2020/036860, filed on Jun. 10, 2020, the entire said invention is incorporated herein by reference).

In some embodiments, the guide RNA is covalently linked to an RNA template comprising a prime binding sequence (PBS) and a reverse transcription template (RTT), and thus is a pegRNA or epegRNA (with an additional stabilizing motif at its 3′-end) used in a prime editor.

In some embodiments, the pegRNA or epegRNA has one or more non-nucleotide linkers (nNt-linker), and thus is a chemically ligated pegRNA or epegRNA.

### Non-Nucleotide Linkers (nNt-Linker)

An nNt-Linker, formed by chemical ligation, comprises an M core structure of Formula M-1 to M-13 as non-limiting examples:

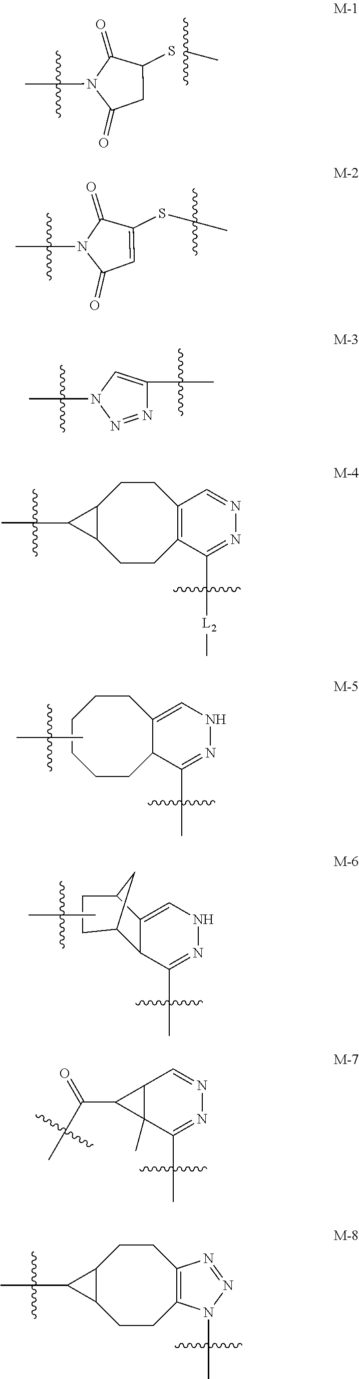

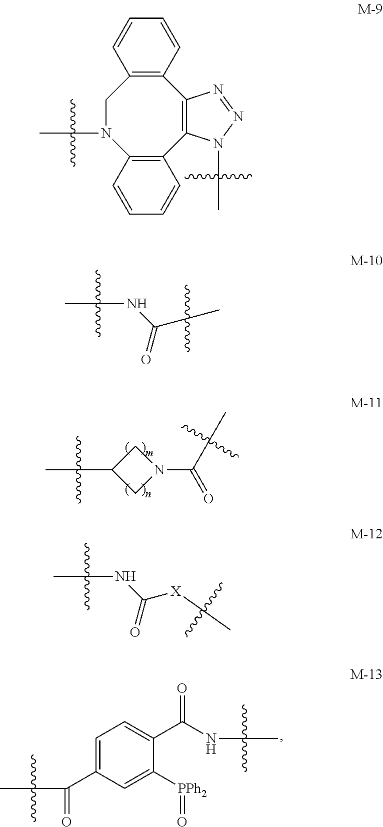

wherein X═O, S, NH, or CH₂, m=0 to 3 and n=0 to 3,

and two L linkers comprising Formula L-1 to L-23 as non-limiting examples:

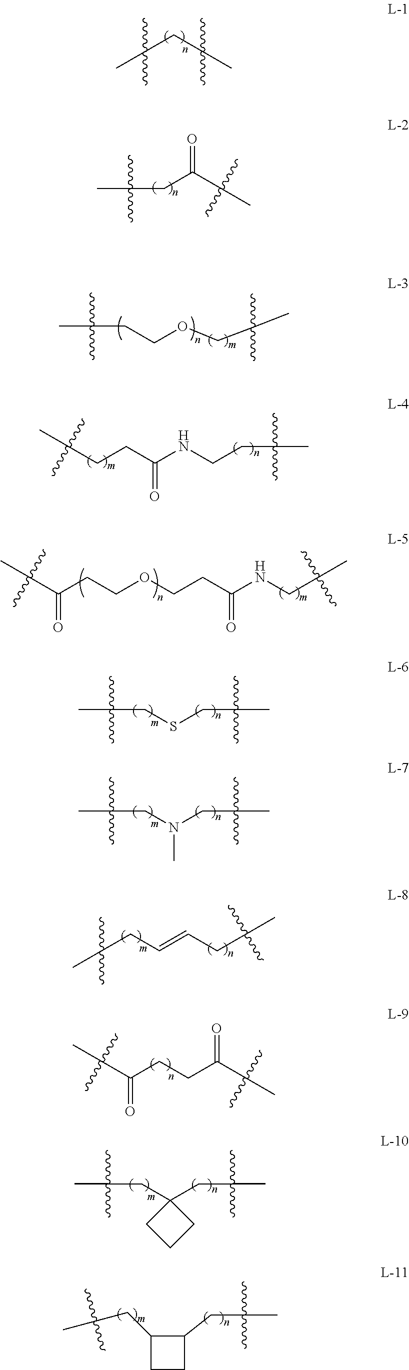

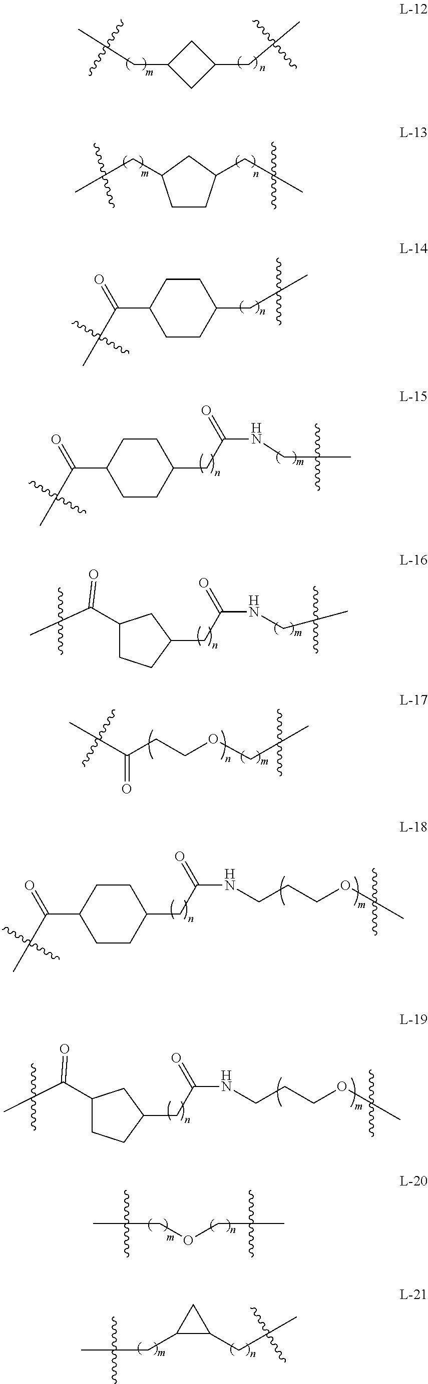

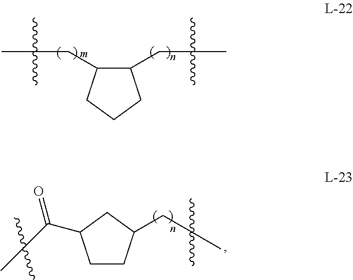

wherein m=0 to 16 and n=0 to 16,

said L linkers and said M core structure are joined as L-M-L, wherein the two L linkers are the same or different, and each L optionally comprises one or more structures of Formula L-1 to L-23 or partial structure(s), and attached to two terminal nucleotides of Formula Nuc-1 to Nuc-18 as non-limiting examples:

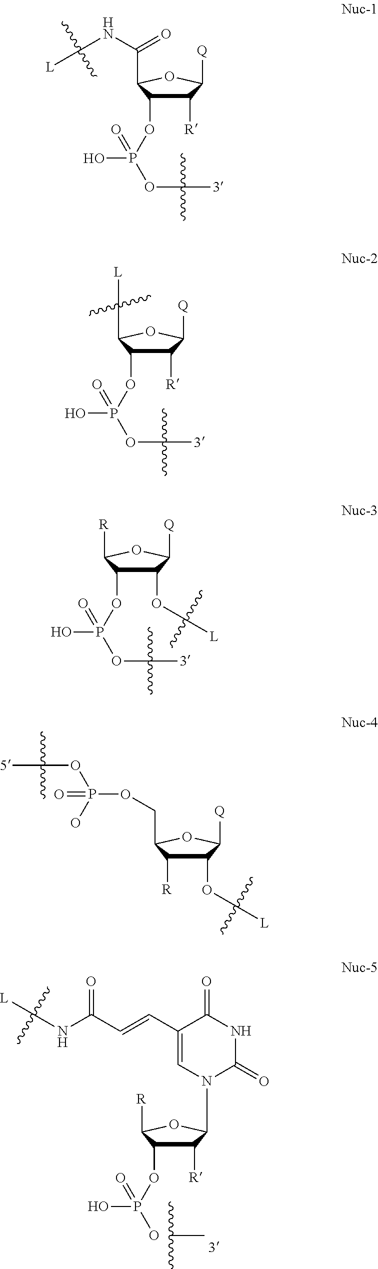

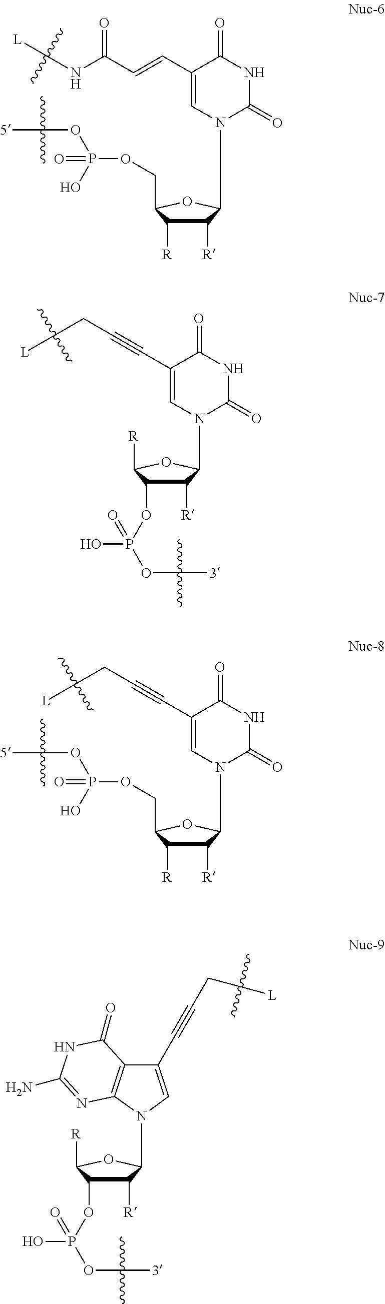

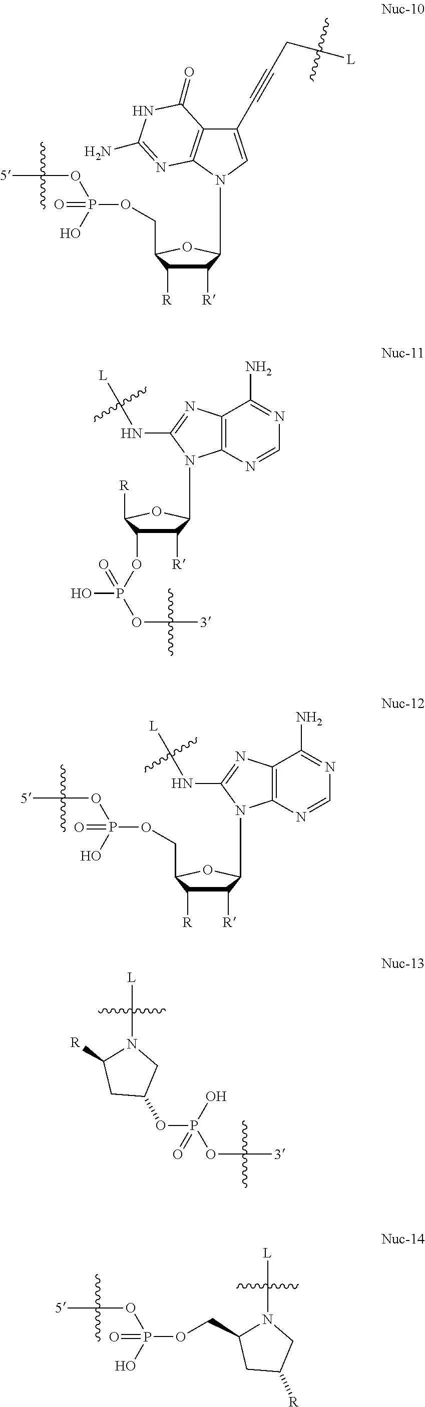

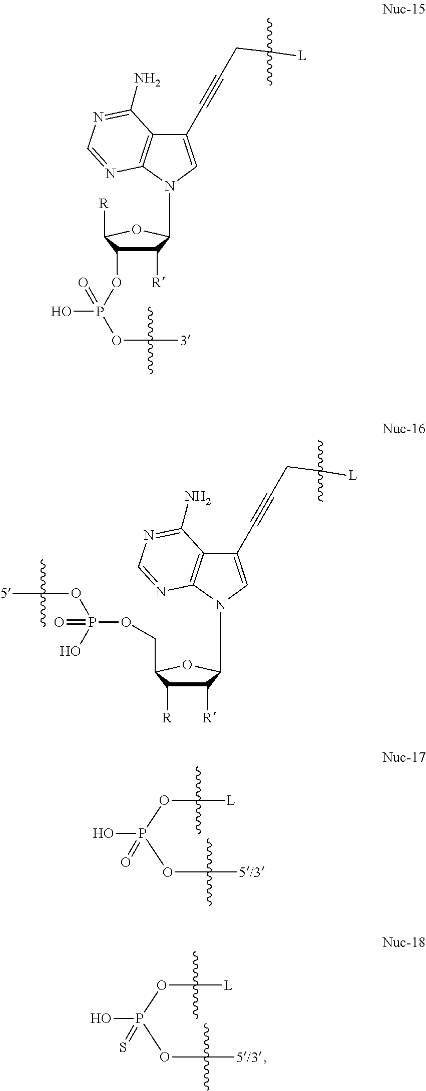

wherein the attached positions are

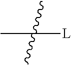

### to L-M-L and

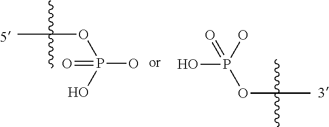 [SMILES: *OP(=O)(O)O ; *OP(=O)(O)O]

to upstream and downstream oligonucleotides, respectively, and wherein R is H, OH,

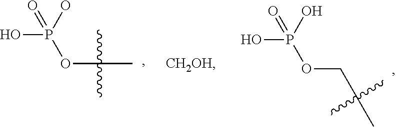 [SMILES: *OP(=O)(O)O ; *COP(=O)(O)O]

F, NH₂, OMe, CH₂OMe, OCH₂CH₂OMe, an alkyl, a cycloalkyl, an aryl, or heteroaryl, R′ is H, OH,

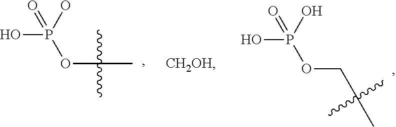 [SMILES: *OP(=O)(O)O ; *COP(=O)(O)O]

F, NH₂, OMe, CH₂OMe, OCH₂CH₂OMe, an alkyl, a cycloalkyl, an aryl, or a heteroaryl, and Q is a natural or a non-natural nucleic acid base.

In some embodiments, the M core structure, L, and terminal nucleotides are optionally modified with substituents such as halogen (F, Cl, Br, I), lower alkyl of C₁-C₆, halogenated (F, Cl, Br, I) lower alkyl of C₁-C₆, lower alkenyl of C₂-C₆, halogenated (F, Cl, Br, I) lower alkenyl of C₂-C₆, CN, lower alkynyl of C₂-C₆, halogenated (F, Cl, Br, I) lower alkynyl of C₂-C₆, lower alkoxy of C₁-C₆, halogenated (F, Cl, Br, I) lower alkoxy of C₁-C₆, an optionally substituted aryl, an optionally substituted heteroaryl, an optionally substituted sulfonyl, or optionally substituted acyl, which includes but is not limited to C(═O) alkyl, NR′₂, CN, CO₂H, CO₂R′, CONH₂, CONHR′, CONR′₂, CH═CHCO₂H, or CH═CHCO₂R′, wherein R′is an optionally substituted alkyl, which includes, but is not limited to, H, an optionally substituted C₁-C₂₀ alkyl, an optionally substituted lower alkyl, an optionally substituted cycloalkyl, an optionally substituted alkynyl of C₂-C₆, an optionally substituted lower alkenyl of C₂-C₆, an optionally substituted aryl, an optionally substituted heteroaryl, an optionally substituted sulfonyl, or optionally substituted acyl, which includes but is not limited to C(═O) alkyl, or alternatively, in the instance of NR′₂, each R′ comprise at least one C atom that are joined to form a heterocycle comprising at least two carbon atoms.

In some embodiments, an nNt-Linker joins the 3′-terminal nucleotide of a crRNA and the 5′-terminal nucleotide of a tracrRNA. In some embodiments, an nNt-Linker joins the 5′-terminal nucleotide of a crRNA and the 3′-terminal nucleotide of a tracrRNA. In some embodiments, an nNt-Linker joins two oligonucleotide segments of tracrRNA.

In some embodiments, one of the two L's in nNt-linkers (L-M-L) is covalently linked to an exposed amino acid residue of AAV capsid protein such as ncAA, lysine, serine, and cysteine, and the other is covalently linked to the guide RNA.

In some embodiments, the nNt-linkers between the two nucleotides/nucleosides are represented by the following formulas:

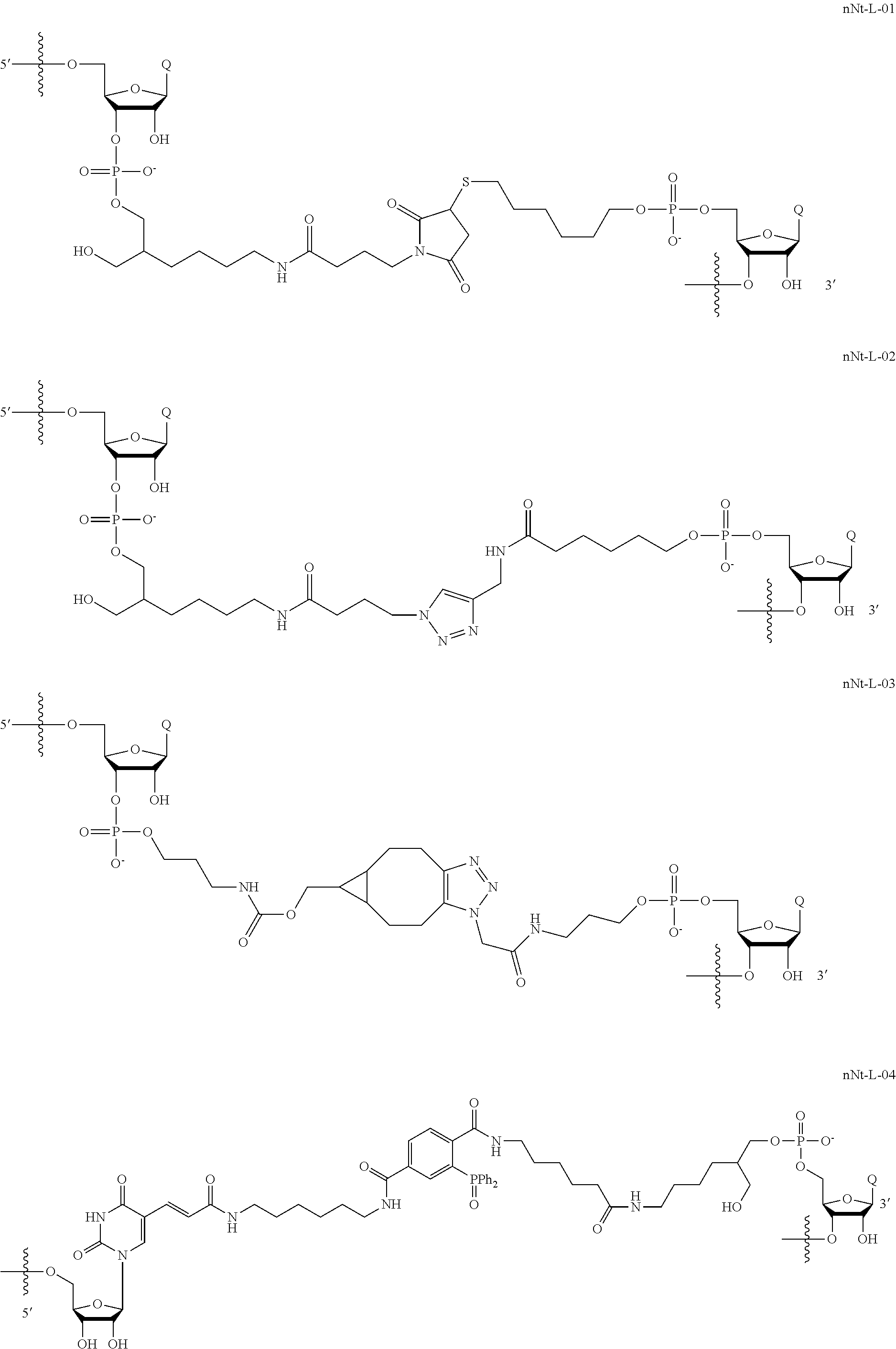

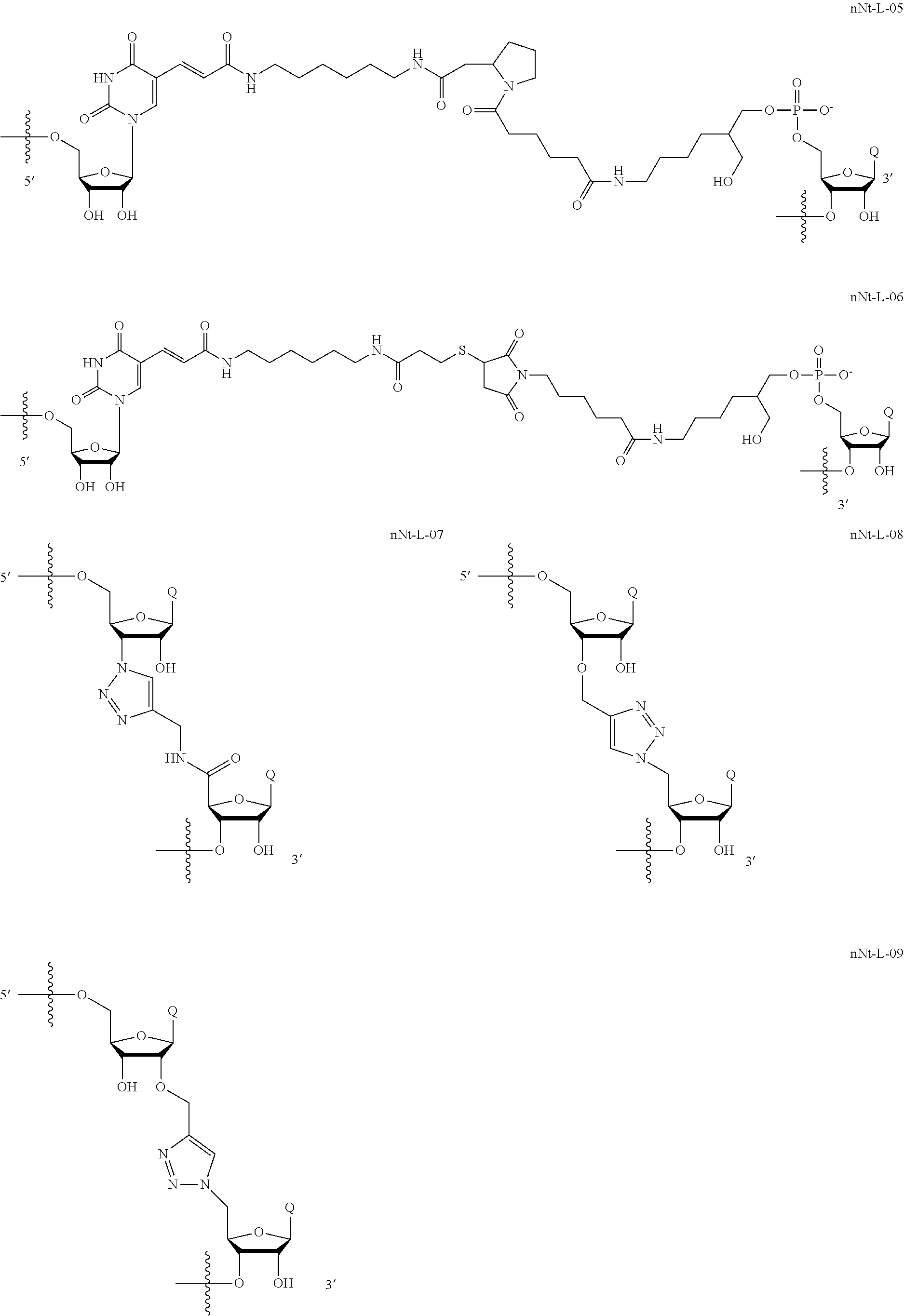

### CRISPR Effector Proteins

In some embodiments, CRISPR effector endonuclease is selected from Cas proteins of Type II, Class 2 including Streptococcus pyogenes-derived Cas9 (SpCas9, 4.1 kb), smaller Cas9 orthologues, including Staphylococcus aureus-derived Cas9 (SaCas9, 3.16 kb), Campylobacter jejuni-derived Cas9 (CjCas9, 2.95 kb), Streptococcus thermophilus Cas9 (St1Cas9, 3.3 kb), Neisseria meningitidis Cas9 (NmCas9, 3.2 kb), and many other variants of engineered Cas9 proteins such as SpCas9-HF1, eSpCas9, and HypaCas9, proteins of Type V, Class 2 including Cas12 (Cas12a (Cpf1), Cas12b (C2c1), Cas 12c, Cas12e, Cas12g, Cas12h, Cas12i, and etc.) and Cas14, and proteins of Type VI, Class 2 such as Cas13a and Cas13b. The said CRISPR effector protein can be a nickase e.g. nCas9 such as a SpCas9-nickase (D10A or H840A), or a catalytically inactive protein e.g. dCas9, coupled/fused with a protein effector such as a DNA polymerase, FokI, transcription activator(s), transcription repressor(s), catalytic domains of DNA methyltransferase, histone acetyltransferase and deacetylase, reverse transcriptase (prime editor), and nucleic acid deaminases (base editor) at its either N- or C-terminal.

In another embodiment, the said CRISPR effector endonuclease is an artificial one comprising one or more functional domains derived from human.

In yet another embodiment, the said CRISPR effector endonuclease is a class 2 CRISPR Cas protein functionalized by site-directed mutagenesis to introduce orthogonal conjugating sites such as cysteines and remove deleterious conjugating sites (e.g. C80 in SpCas9), and corresponding RNP conjugates are prepared by selective conjugations such as PEGylation of cysteines by maleimide chemistry.

In yet another embodiment, the said CRISPR effector endonuclease is a class 2 CRISPR Cas protein fused with a human DNA or RNA polymerase via a peptide linker.

In some embodiments, the CRISPR-Cas virus-RNP conjugate is covalently linked by a peptide linker joining the viral capsid protein and a Cas protein, i.e., a Cas-capsid fusion protein. The guide RNA conjugates with the virus via its binding to the Cas protein, wherein the Cas protein is optionally fused with another effector protein (e.g., a DNA directed DNA polymerase, a reverse transcriptase, and etc.).

In some embodiments, the CRISPR-Cas virus-RNP conjugate is covalently linked by a peptide linker joining the viral capsid protein and a Cas protein or a Cas fusion protein, wherein the peptide linker is flexible and of 10-50 amino acids in length (e.g., VP1-linker-Cas9_H840A-linker-DNA_pol) and any two peptide linkers can be the same or different.

In some embodiments, the Cas-capsid fusion protein is encoded by a DNA (e.g., a plasmid and a viral vector) and the peptide linker is located between the C-terminus of capsid protein and the N-terminus of the Cas protein or the Cas fusion protein, e.g., the Cas protein is optionally fused with an effector protein such as a DNA-directed DNA polymerase.

In some embodiments, the Cas-capsid fusion protein is encoded by an mRNA and the peptide linker is located between the C-terminus of capsid protein and the N-terminus of the Cas protein or the Cas fusion protein, e.g., the Cas protein is optionally fused with an effector protein such as a DNA-directed DNA polymerase.

### Tissue Tropic Viral Vectors

In some embodiments, the said conjugated viral vector is a retrovirus, lentivirus, adenovirus, AAV, or baculovirus.

In some embodiments, the said viral vector is an engineered AAV or AAV chimera to enable high transduction efficiency at a targeted tissue by changing the tropism of AAV capsids and to have low immunogenicity by evading human preexisting anti-AAV capsid neutralizing antibodies.

In some embodiments, the said viral vector is a native or an engineered AAV to enable brain tissue targeted delivery. Such AAV serotypes include as non-limiting examples AAV1, AAV2/DJ, AAV2/DJ8, AAV2g9, AAV2-retro and scAAV9.

In some embodiments, the said viral vector is a native or an engineered AAV to enable liver tissue targeted delivery. Such AAV serotypes include as non-limiting examples AAV8 and AAV3.

In some embodiments, the said viral vector is a native or an engineered AAV to enable muscle tissue targeted delivery. Such AAV serotypes include as non-limiting examples AAV6, AAV8 and AAV9.

In some embodiments, the said viral vector is a native or an engineered AAV to enable heart tissue targeted delivery. Such AAV serotypes include as non-limiting examples AAV rh74 and AAV9.

In some embodiments, the said viral vector is a native or an engineered AAV to enable retina tissue targeted delivery. Such AAV serotypes include as non-limiting examples AAV1, AAV2, AAV5, AAV8 and AAV9.

In some embodiments, the said viral is a native or an engineered AAV to enable lung tissue targeted delivery. Such AAV serotypes include as non-limiting examples AAV9.

In some embodiments, the expression of a transgene packaged in the said viral vector is driven by inducible tissue-specific promoters.

In some embodiments, the expression of a transgene packaged in the said viral vector, is driven by brain tissue-specific promoters such as pMecp2, hSyn1, TRE3G and EFS as non-limiting examples.

In some embodiments, the expression of a transgene packaged in the said viral vector, is driven by liver tissue-specific promoters such as TBG and HCRhAATp or by lung tissue-specific promoters such as EFS, as non-limiting examples.

In some embodiments, the expression of a transgene packaged in the said viral vector, is driven by heart tissue-specific promoters such as CMV, Myh6, CB and CK7-miniCMV as non-limiting examples.

In some embodiments, the expression of a transgene packaged in the said viral vector, is driven by retina tissue-specific promoters such as EFS, CMV, Spc512, pMecp2, and Picam2 as non-limiting examples.

In some embodiments, the expression of a transgene packaged in the said viral vector, is driven by muscle tissue-specific promoters such as CMV, EFS and CK8 as non-limiting examples.

In some embodiments, the guide RNA-viral vector conjugate is packaged with a transgene encoding a functional protein to treat human single-gene disorders, or multiple functional proteins joined by cleavable peptide linkers to treat human polygenic disorders.

In some embodiments, the guide RNA-viral vector conjugate can be multiplexed to produce multiple functional proteins in cells to treat human polygenic disorders.

Non-limiting examples of such human single-gene disorders are given below (Table 1). Other examples include human polygenic disorders such as heart disease and diabetes.

TABLE 1
Examples single-gene disorders and their prevalence

| | |
|---|---|
| | Prevalence |
| Disorder | (approximate) |

Autosomal dominant

| | |
|---|---|
| Familial hypercholesterolemia | 1 in 500 |
| Polycystic kidney disease | 1 in 1250 |
| Neurofibromatosis type I | 1 in 2,500 |
| Hereditary spherocytosis | 1 in 5,000 |
| Marfan syndrome | 1 in 4,000 |
| Huntington's disease | 1 in 15,000 |

Autosomal recessive

| | |
|---|---|
| Sickle cell anaemia | 1 in 625 |
| Cystic fibrosis | 1 in 2,000 |
| Tay-Sachs disease | 1 in 3,000 |
| Phenylketonuria | 1 in 12,000 |
| Mucopolysaccharidoses | 1 in 25,000 |
| Lysosomal acid lipase deficiency | 1 in 40,000 |
| Glycogen storage diseases | 1 in 50,000 |

X-linked

| | |
|---|---|
| Duchenne muscular dystrophy | 1 in 7,000 |
| Hemophilia | 1 in 10,000 |

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure.

Further, the dates of publication provided may be different from the actual publication dates that may need to be independently confirmed.

This disclosure is not limited to particular embodiments described, as such may vary. It is also to be understood that the terminology used herein is for the purpose of describing particular embodiments only, and is not intended to be limiting, since the scope of the present disclosure will be limited only by the appended claims.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### EXAMPLES

The following examples further illustrate embodiments of the disclosed invention, which are not limited by these examples.

### Example 1: crgRNA-AAVS1

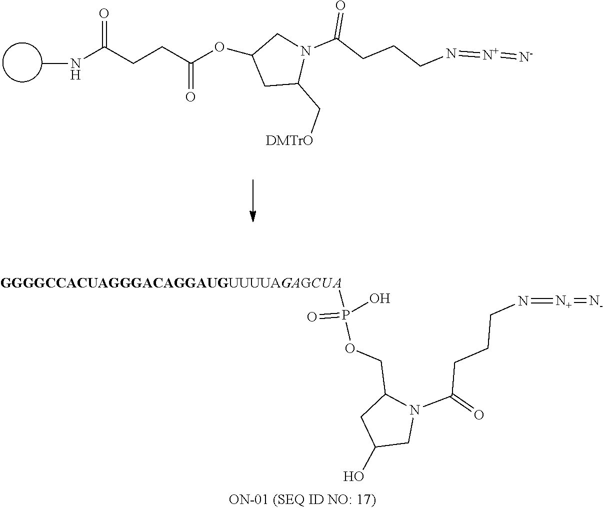

ON-01 was prepared on an Expedite 8909 automated DNA/RNA synthesizer using the standard 1.0 μmole RNA phosphoramidite cycle. 3′-Azido CPG 1000 Å (1 μmole) was packed into an Expedite column. All β-cyanoethyl phosphoramidite monomers were dissolved in anhydrous acetonitrile to a concentration of 0.1 M immediately prior to use. Coupling, capping and oxidation reagents (ChemGenes) were 5-Ethyl-1H-tetrazole (0.45 M in acetonitrile), Cap A (10% N-Methylimidazole in THF)/Cap B (10% N-Methylimidazole in THF) and iodine (0.02M Iodine/Pyridine/H₂O/THF), respectively. Stepwise coupling efficiencies were determined by automated trityl cation conductivity monitoring and in all cases were >97%.

Oligonucleotide on solid support was treated with 20% piperidine in DMF at room temperature to suppress the formation of cyanoethyl adducts, then washed with acetonitrile (3×1 mL) and dried with argon.

RNA deprotection. The oligonucleotide on solid support was exposed to AMA (Ammonium Hydroxide/40% aqueous Methylamine 1:1 v/v) in a sealed vial for 20 min at 65° C. The solution was collected by filtration and the solution was then concentrated till dryness in a Savant SpeedVac concentrator at room temperature. The resulting white solid was re-dissolved in a 2:2:3 v/v mixture of dry NMP (200 μL), triethylamine (200 μL) and triethylamine trihydrofluoride (300 μL) and heated at 60° C. for 3 h. After cooling down to room temperature, sodium acetate (3M pH 5.2, 40 μL) and ethanol (1 mL) were added and the RNA was stored for 30 min at −78° C. The RNA was then pelleted by centrifugation (15,850×g, 10 min, 4° C.), the supernatant discarded and the pellet washed twice with 70% ethanol (500 μL). The pellet was then dried in vacuo and used for next step without further purification.

CrgRNA-AAVS1 modified with one or more G-clamp nucleotides were synthesized using a G-clamp phosphoramidite.

### Example 2: 3′-Amino Modified tracrgRNA

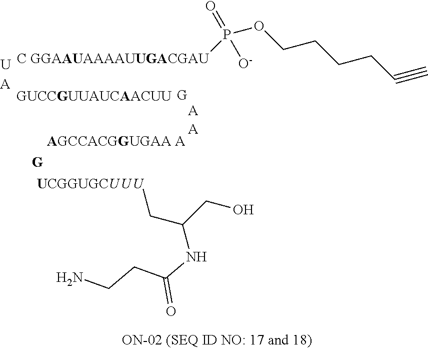

ON-02 was prepared on an Expedite 8909 automated DNA/RNA synthesizer using the standard 1.0 μmole RNA phosphoramidite cycle, fully deprotected and separated as ON-01. 3′-amino modifier lcaa CPG 1000 Å (1 μmole) was used instead. The pellet was then dried in vacuo and used for next step without further purification.

### Example 3: 3′-Azido modified LgRNA-AAVS1

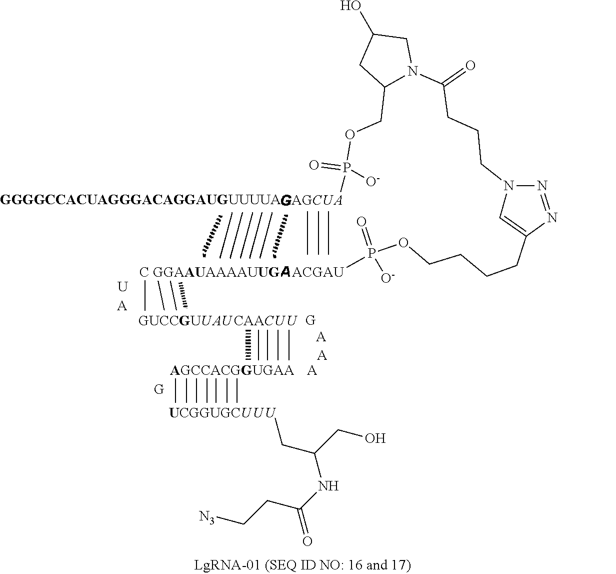

To azide ON-1 pellet (half, <0.49 μmole) and alkyne ON-2 pellet (half, <0.49 μmole) in a stock solution (DMSO/ddH₂O/2 M TEAA, 2:1:0.4, 1700 μL) is added CuSO₄-THPTA (tris-hydroxypropyl triazole ligand) (250 mM, 100 μL), and the resulting light blue solution is deoxygenated by bubbling argon for 10 min. Freshly prepared ascorbic acid in ddH₂O (125 mM, 200 μL) is added, and reaction mixture is further deoxygenated by bubbling argon for 30 min. The reaction mixture was sealed and kept at room temperature for 2 h, and sodium acetate (3 M pH 5.2, 40 μL) and ethanol (1 mL) were added. The resulting RNA suspension is stored for 30 min at −78° C. The RNA is then pelleted by centrifugation (15,850×g, 10 min, 4° C.). The supernatant is discarded and the pellet washed twice with 70% ethanol (500 μL). The pellet is then dried in vacuo at room temperature.

The above oligonucleotide pellet is mixed with gel loading buffer (formamide/ddH₂O 90% v/v, with 10 mM EDTA) and RNA loading dyes (2×) and loaded onto a denaturing 10% polyacrylamide gel (1× TBE buffer containing 7M urea) and separated at 65 W for 2-3 h. RNA bands are visualized under UV, excised, crushed, soaked in a gel extract buffer (NaCl solution with 2 mM EDTA) overnight at 37° C. with vigorous shaking. The gel is removed by filtration through two consecutive Sep-Pak C18 plus short cartridges, the oligonucleotide solutions are combined, and the final concentration is determined by a NanoDrop spectrophotometer at 260 nm. The solution is concentrated till dryness in vacuo in a Savant SpeedVac concentrator at room temperature to give the 3′-amino modified product.

The above product is transformed to 3′-azido modified LgRNA by either a reaction with an azido substituted NHS ester or a diazo transfer reaction with reagents such as fluorosulfuryl azide (FSO₂N₃).

### Example 7: In Vitro Cleavage Assay

Recombinant Cas9 protein was purchased from New England BioLabs, Inc. Cas9 and lgRNA were preincubated in a 1:1 molar ratio in the cleavage buffer to reconstitute the RNP complex.

The substrate of a dsDNA comprising AAVS1 site was dissolved in the cleavage buffer and added to the RNP complex. The reaction mixture was incubated at 37° C. for 1 h, and DNA loading dyes (6×) was added. The resulting mixture was heated at 95° C. for 5 min, cooled to room temperature, and resolved by a 1% Agarose gel.

### Example 8: Plasmid for AAV Capsid Engineered with ncAA for Conjugations

A UAG stop codon is placed in the Cap gene at a site (e.g., T456) to enable the incorporation of ncAAs. To limit the copies of conjugated guide RNAs, ncAA is selectively introduced into VP1 or VP2 only. Plasmids (pIDTsmart-RC2-AVP1-CMV-VP1 (TAG) or pIDTsmart-RC2-AVP2-CMV-VP2 (TAG)) for producing AAVs with ncAA incorporated at selected capsid proteins are designed as reported in literature with modifications (Chatterjee, et al. Bioconjug Chem. 2024, 35, 64-71.).

The translation start codon(s) of VP1 and/or VP2 (ΔVP1, ΔVP2, or ΔVP1,2) are mutated to selectively abolish the expression of these proteins from Cap. The deleted VP1 or VP2 is supplied back in trans from a separate Cap gene driven by a CMV promotor, wherein VP3 expression is eliminated by mutating its translation start sites (ΔVP3) and a UAG codon is incorporated at the T456 position.

### Example 9: LgRNA-AAV Conjugate Joined at ncAA Site in VP1 or VP2 and its RNP Complex

Plasmids for producing ncAA containing AAVs are described as follows. pHelper plasmid contains the adenoviral E2A, E4, and VA genes. pIDTSmart-MbPyIRS-8xPytR-ITR-transgene plasmid contains wild-type M. barkeri pyrrolysyl synthetase driven by a CMV promotor, eight copies of the M. mazei pyrrolysyl tRNACUA expression cassette driven by a human U6 promoter, and a CMV-transgene cargo flanked by packaging signals (AAV ITRs). pIDTsmart-RC2-ΔVP1-CMV-VP1(TAG) or pIDTsmart-RC2-ΔVP2-CMV-VP2(TAG) contains the cap and rep gene.

AAV is produced by transfecting HEK293T cells with the above plasmids with a routine protocol at the presence of BCNK.

BCNK-containing AAV preparations are conjugated with 20 μM 3′-azido LgRNA for varying amounts of time at room temperature.

The resulting LgRNA-AAV conjugate is incubated with Cas9 protein in a 1:1 molar ratio in the cleavage buffer to reconstitute the RNP complex.

### Example 10: LgRNA-AAV Conjugates Further Modified at Exposed Lysine Residues and its and its RNP Complex

The above LgRNA-AAV conjugate are conjugated with 20 μM NCS-modified AAV capsid modifier for varying amounts of time at room temperature. The capsid modifier is selected from cell-targeting ligands (e.g., GalNAc), peptides, aptamers, PEG and etc.

The resulting LgRNA-AAV-ligand conjugates are incubated with Cas9 protein in a 1:1 molar ratio in the cleavage buffer to reconstitute the RNP complex.

### EQUIVALENTS

The present disclosure is not to be limited in terms of the particular embodiments described in this application, which are intended as single illustrations of individual aspects of the present disclosure. Many modifications and variations of this present disclosure can be made without departing from its spirit and scope, as will be apparent to those skilled in the art. Functionally equivalent methods and apparatuses within the scope of the present disclosure, in addition to those enumerated herein, will be apparent to those skilled in the art from the foregoing descriptions. Such modifications and variations are intended to fall within the scope of the present disclosure. It is to be understood that this present disclosure is not limited to particular methods, reagents, compounds compositions, or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 or 1 to 3 items refers to groups having 1, 2, or 3 items. Similarly, a group having 1-5 or 1 to 5 items refers to groups having 1, 2, 3, 4, or 5 items, and so forth.

All patents, patent applications, provisional applications, and publications referred to or cited herein are incorporated by reference in their entirety, including all figures and tables, to the extent they are not inconsistent with the explicit teachings of this specification.

### AAV2 Plasmids

pIDTsmart-RC2-ΔVP1-CMV-VP1(TAG) (T454X, T455X, or T456X)
X = ncAA (T456X is listed in the sequence below)
(SEQ ID NO: 20)
cccgtgtaaaacgacggccagtttatctagtcagcttgattctagctgatcgtggaccggaaggtgagccagtgagttgattgcagtccagttacgctg
gagtctgaggctcgtcctgaatgatatgcgaccgccggagggttgcgtttgagacgggcgacagatccagtcgcgctgctctcgtcgatccgctagggc
ggccgctctagaactagtggatcccccggaagatcagaagttcctattccgaagttcctattctctagaaagtataggaacttctgatctgcgcagccg
ccatgccggggttttacgagattgtgattaaggtccccagcgaccttgacgagcatctgcccggcatttctgacagctttgtgaactgggtggccgaga
aggaatgggagttgccgccagattctgacatggatctgaatctgattgagcaggcacccctgaccgtggccgagaagctgcagcgcgactttctgacgg
aatggcgccgtgtgagtaaggccccggaggcccttttctttgtgcaatttgagaagggagagagctacttccacatgcacgtgctcgtggaaaccaccg
gggtgaaatccatggttttgggacgtttcctgagtcagattcgcgaaaaactgattcagagaatttaccgcgggatcgagccgactttgccaaactggt
tcgcggtcacaaagaccagaaatggcgccggaggcgggaacaaggtggtggatgagtgctacatccccaattacttgctccccaaaacccagcctgagc
tccagtgggcgtggactaatatggaacagtatttaagcgcctgtttgaatctcacggagcgtaaacggttggtggcgcagcatctgacgcacgtgtcgc
agacgcaggagcagaacaaagagaatcagaatcccaattctgatgcgccggtgatcagatcaaaaacttcagccaggtacatggagctggtcgggtggc
tcgtggacaaggggattacctcggagaagcagtggatccaggaggaccaggcctcatacatctccttcaatgcggcctccaactcgcggtcccaaatca
aggctgccttggacaatgcgggaaagattatgagcctgactaaaaccgcccccgactacctggtgggccagcagcccgtggaggacatttccagcaatc
ggatttataaaattttggaactaaacgggtacgatccccaatatgcggcttccgtctttctgggatgggccacgaaaaagttcggcaagaggaacacca
tctggctgtttgggcctgcaactaccgggaagaccaacatcgcggaggccatagcccacactgtgcccttctacgggtgcgtaaactggaccaatgaga
actttcccttcaacgactgtgtcgacaagatggtgatctggtgggaggaggggaagatgaccgccaaggtcgtggagtcggccaaagccattctcggag
gaagcaaggtgcgcgtggaccagaaatgcaagtcctcggcccagatagacccgactcccgtgatcgtcacctccaacaccaacatgtgcgccgtgattg
acgggaactcaacgaccttcgaacaccagcagccgttgcaagaccggatgttcaaatttgaactcacccgccgtctggatcatgactttgggaaggtca
ccaagcaggaagtcaaagactttttccggtgggcaaaggatcacgtggttgaggtggagcatgaattctacgtcaaaaagggtggagccaagaaaagac
ccgcccccagtgacgcagatataagtgagcccaaacgggtgcgcgagtcagttgcgcagccatcgacgtcagacgcggaagcttcgatcaactacgcag
acaggtaccaaaacaaatgttctcgtcacgtgggcatgaatctgatgctgtttccctgcagacaatgcgagagaatgaatcagaattcaaatatctgct
tcactcacggacagaaagactgtttagagtgctttcccgtgtcagaatctcaacccgtttctgtcgtcaaaaaggcgtatcagaaactgtgctacattc
atcatatcatgggaaaggtgccagacgcttgcactgcctgcgatctggtcaatgtggatttggatgactgcatctttgaacaataaatgatttaaatca
ggtCTCgctgccgatggttatcttccagattggctcgaggacactctctctgaaggaataagacagtggtggaagctcaaacctggcccaccaccacca
aagcccgcagagcggcataaggacgacagcaggggtcttgtgcttcctgggtacaagtacctcggacccttcaacggactcgacaagggagagccggtc
aacgaggcagacgccgcggccctcgagcacgacaaagcctacgaccggcagctcgacagcggagacaacccgtacctcaagtacaaccacgccgacgcg
gagtttcaggagcgccttaaagaagatacgtcttttgggggcaacctcggacgagcagtcttccaggcgaaaaagagggttcttgaacctctgggcctg
gttgaggaacctgttaagacggctccgggaaaaaagaggccggtagagcactctcctgtggagccagactcctcctcgggaaccggaaaggcgggccag
cagcctgcaagaaaaagattgaattttggtcagactggagacgcagactcagtacctgacccccagcctctcggacagccaccagcagccccctctggt
ctgggaactaatacgatggctacaggcagtggcgcaccaatggcagacaataacgagggcgccgacggagtgggtaattcctcgggaaattggcattgc
gattccacatggatgggcgacagagtcatcaccaccagcacccgaacctgggccctgcccacctacaacaaccacctctacaaacaaatttccagccaa
tcaggagcctcgaacgacaatcactactttggctacagcaccccttgggggtattttgacttcaacagattccactgccacttttcaccacgtgactgg
caaagactcatcaacaacaactggggattccgacccaagagactcaacttcaagctctttaacattcaagtcaaagaggtcacgcagaatgacggtacg
acgacgattgccaataaccttaccagcacggttcaggtgtttactgactcggagtaccagctcccgtacgtcctcggctcggcgcatcaaggatgcctc
ccgccgttcccagcagacgtcttcatggtgccacagtatggatacctcaccctgaacaacgggagtcaggcagtaggacgctcttcattttactgcctg
gagtactttccttctcagatgctgcgtaccggaaacaactttaccttcagctacacttttgaggacgttcctttccacagcagctacgctcacagccag
agtctggaccgtctcatgaatcctctcatcgaccagtacctgtattacttgagcagaacaaacactccaagtggaaccaccacgcagtcaaggcttcag
ttttctcaggccggagcgagtgacattcgggaccagtctaggaactggcttcctggaccctgttaccgccagcagcgagtatcaaagacatctgcggat
aacaacaacagtgaatactcgtggactggagctaccaagtaccacctcaatggcagagactctctggtgaatccgggcccggccatggcaagccacaag
gacgatgaagaaaagttttttcctcagagcggggtctcatctttgggaagcaaggctcagagaaaacaaatgtggacattgaaaaggtcatgattacag
acgaagaggaaatcaggacaaccaatcccgtggctacggagcagtatggttctgtatctaccaacctccagagaggcaacagacaagcagctaccgcag
atgtcaacacacaaggcgttcttccaggcatggtctggcaggacagagatgtgtaccttcaggggcccatctgggcaaagattccacacacggacggac
attttcacccctctcccctcatgggggattcggacttaaacaccctcctccacagattctcatcaagaacaccccggtacctgcgaatccttcgaccac
cttcagtgcggcaaagtttgcttccttcatcacacagtactccacgggacaggtcagcgtggagatcgagtgggagctgcagaaggaaaacagcaaacg
ctggaatcccgaaattcagtacacttccaactacaacaagtctgttaatgtggactttactgtggacactaatggcgtgtattcagagcctcgccccat
tggcaccagatacctgactcgtaatctgtaattgcttgttaatcaataaaccgtttaattcgtttcagttgaactttggtctctgcgtatttctttctt
atctagtttccatggctacgtagataagtagcatggcgggttaatcattaactacagcccgggcgtttaaacagcgggcggaggggtggagtcgtgacg
tgaattacgtcatagggttagggaggtcctgtattagaggtcacgtgagtgttttgcgacattttgcgacaccatgtggtctcgctgggggggggggcc
cgagtgagcacgcagggtctccattttgaagcgggaggtttgaacgagcgctggcgcgctcactggccgtcgttttacaacgtcgtgactgggaaaacc
ctggcgttacccaacttaatcgccttgcagcacatccccctttcgccagctggcgtaatagcgaagaggcccgcaccgatcgcccttcccatgcatcgg
ccgcaaatacctgcaggatccgttttgcgctgcttcgcgatgtacgggccagatatacgcgttgacattgattattgactagttattaatagtaatcaa
ttacggggtcattagttcatagcccatatatggagttccgcgttacataacttacggtaaatggcccgcctggctgaccgcccaacgacccccgcccat
tgacgtcaataatgacgtatgttcccatagtaacgccaatagggactttccattgacgtcaatgggtggactatttacggtaaactgcccacttggcag
tacatcaagtgtatcatatgccaagtacgccccctattgacgtcaatgacggtaaatggcccgcctggcattatgcccagtacatgaccttatgggact
ttcctacttggcagtacatctacgtattagtcatcgctattaccatggtgatgcggttttggcagtacatcaatgggcgtggatagcggtttgactcac
ggggatttccaagtctccaccccattgacgtcaatgggagtttgttttggcaccaaaatcaacgggactttccaaaatgtcgtaacaactccgccccat
tgacgcaaatgggggtaggcgtgtacggtgggaggtctatataagcagagctctctggctaactagagaacccactgcttactggcttatcgaaattaa
tacgactcactatagggagacccaagctggctagcatggctgccgatggttatcttccagattggctcgaggacactctctctgaaggaataagacagt
ggtggaagctcaaacctggcccaccaccaccaaagcccgcagagcggcataaggacgacagcaggggtcttgtgcttcctgggtacaagtacctcggac
ccttcaacggactcgacaagggagagccggtcaacgaggcagacgccgcggccctcgagcacgacaaagcctacgaccggcagctcgacagcggagaca
acccgtacctcaagtacaaccacgccgacgcggagtttcaggagcgccttaaagaagatacgtcttttgggggcaacctcggacgagcagtcttccagg
cgaaaaagagggttcttgaacctctgggcctggttgaggaacctgttaagACCgctccgggaaaaaagaggccggtagagcactctcctgtggagccag
actcctcctcgggaaccggaaagggggccagcagcctgcaagaaaaagattgaattttggtcagactggagacgcagactcagtacctgacccccagcc
tctcggacagccaccagcagccccctctggtctCggaactaatacCCTCgctacaggcagtggcgcaccaCTCgcagacaataacgagggcgccgacgg
agtgggtaattcctcgggaaattggcattgcgattccacatggCTCggcgacagagtcatcaccaccagcacccgaacctgggccctgcccacctacaa
caaccacctctacaaacaaatttccagccaatcaggagcctcgaacgacaatcactactttggctacagcaccccttgggggtattttgacttcaacag
attccactgccacttttcaccacgtgactggcaaagactcatcaacaacaactggggattccgacccaagagactcaacttcaagctctttaacattca
agtcaaagaggtcacgcagaatgacggtacgacgacgattgccaataaccttaccagcacggttcaggtgtttactgactcggagtaccagctcccgta
cgtcctcggctcggcgcatcaaggatgcctcccgccgttcccagcagacgtcttcatggtgccacagtatggatacctcaccctgaacaacgggagtca
ggcagtaggacgctcttcattttactgcctggagtactttccttctcagatgctgcgtaccggaaacaactttaccttcagctacacttttgaggacgt
tcctttccacagcagctacgctcacagccagagtctggaccgtctcatgaatcctctcatcgaccagtacctgtattacttgagcagaacaaacactcc
aagtggaaccacc [symbol] cagtcaaggcttcagttttctcaggccggagcgagtgacattcgggaccagtctaggaactggcttcctggaccctgttaccgc
cagcagcgagtatcaaagacatctgcggataacaacaacagtgaatactcgtggactggagctaccaagtaccacctcaatggcagagactctctggtg
aatccgggcccggccatggcaagccacaaggacgatgaagaaaagttttttcctcagagcggggttctcatctttgggaagcaaggctcagagaaaaca
aatgtggacattgaaaaggtcatgattacagacgaagaggaaatcaggacaaccaatcccgtggctacggagcagtatggttctgtatctaccaacctc
cagagaggcaacagacaagcagctaccgcagatgtcaacacacaaggcgttcttccaggcatggtctggcaggacagagatgtgtaccttcaggggccc
atctgggcaaagattccacacacggacggacattttcacccctctcccctcatgggtggattcggacttaaacaccctcctccacagattctcatcaag
aacaccccggtacctgcgaatccttcgaccaccttcagtgcggcaaagtttgcttccttcatcacacagtactccacgggacaggtcagcgtggagatc
gagtgggagctgcagaaggaaaacagcaaacgctggaatcccgaaattcagtacacttccaactacaacaagtctgttaatgtggactttactgtggac
actaatggcgtgtattcagagcctcgccccattggcaccagatacctgactcgtaatctgtaattgcttgttaatcaataaaccgtttaattcgtttca
gttgaactttggtctctgcgtatttctttcttatctagtttccatggctacgtagataagtagcatggcgggttaatcattaactacagccctaggggt
gcgagcggatcgagcagtgtcgatcactactggaccgcgagctgtgctgcgacccgtgatcttacggcattatacgtatgatcggtccacgatcagcta
gattatctagtcagcttgatgtcatagctgtttcctgaggctcaatactgaccatttaaatcatacctgacctccatagcagaaagtcaaaagcctccg
accggaggcttttgacttgatcggcacgtaagaggttccaactttcaccataatgaaataagatcactaccgggcgtattttttgagttatcgagattt
tcaggagctaaggaagctaaaatgagccatattcaacgggaaacgtcttgcttgaagccgcgattaaattccaacatggatgctgatttatatgggtat
aaatgggctcgcgataatgtcgggcaatcaggtgcgacaatctatcgattgtatgggaagcccgatgcgccagagttgtttctgaaacatggcaaaggt
agcgttgccaatgatgttacagatgagatggtcaggctaaactggctgacggaatttatgcctcttccgaccatcaagcattttatccgtactcctgat
gatgcatggttactcaccactgcgatcccagggaaaacagcattccaggtattagaagaatatcctgattcaggtgaaaatattgttgatgcgctggca
gtgttcctgcgccggttgcattcgattcctgtttgtaattgtccttttaacggcgatcgcgtatttcgtctcgctcaggcgcaatcacgaatgaataac
ggtttggttggtgcgagtgattttgatgacgagcgtaatggctggcctgttgaacaagtctggaaagaaatgcataaactcttgccattctcaccggat
tcagtcgtcactcatggtgatttctcacttgataaccttatttttgacgaggggaaattaataggttgtattgatgttggacgagtcggaatcgcagac
cgataccaggatcttgccatcctatggaactgcctcggtgagttttctccttcattacagaaacggctttttcaaaaatatggtattgataatcctgat
atgaataaattgcagtttcacttgatgctcgatgagtttttctaatgaggacctaaatgtaatcacctggctcaccttcggggggcctttctgcgttgc
tggcgtttttccataggctccgcccccctgacgagcatcacaaaaatcgatgctcaagtcagaggtggcgaaacccgacaggactataaagataccagg
cgtttccccctggaagctccctcgtgcgctctcctgttccgaccctgccgcttaccggatacctgtccgcctttctcccttcgggaagcgtggcgcttt
ctcatagctcacgctgtaggtatctcagttcggtgtaggtcgttcgctccaagctgggctgtgtgcacgaaccccccgttcagcccgaccgctgcgcct
tatccggtaactatcgtcttgagtccaacccggtaagacacgacttatcgccactggcagcagccactggtaacaggattagcagagcgaggtatgtag
gcggtgctacagagttcttgaagtggtggcctaactacggctacactagaagaacagtatttggtatctgcgctctgctgaagccagttacctcggaaa
aagagttggtagctcttgatccggcaaacaaaccaccgctggtagcggtggtttttttgtttgcaagcagcagattacgcgcagaaaaaaaggatctca
agaagatcctttgattttctaccgaagaaaggccca
pIDTsmart-RC2-ΔVP2-CMV-VP2(TAG) (T454X, T455X, or T456X)
X = ncAA (T456X is listed in the sequence below)
(SEQ ID NO: 21)
cccgtgtaaaacgacggccagtttatctagtcagcttgattctagctgatcgtggaccggaaggtgagccagtgagttgattgcagtccagttacgctg
gagtctgaggctcgtcctgaatgatatgcgaccgccggagggttgcgtttgagacgggcgacagatccagtcgcgctgctctcgtcgatccgctagggc
ggccgctctagaactagtggatcccccggaagatcagaagttcctattccgaagttcctattctctagaaagtataggaacttctgatctgcgcagccg
ccatgccgggg////ttttacgagattgtgattaaggtccccagcgaccttgacgagcatctgcccggcatttctgacagctttgtgaactgggtggcc
gagaaggaatgggagttgccgccagattctgacatggatctgaatctgattgagcaggcacccctgaccgtggccgagaagctgcagcgcgactttctg
acggaatggcgccgtgtgagtaaggccccggaggcccttttctttgtgcaatttgagaagggagagagctacttccacatgcacgtgctcgtggaaacc
accggggtgaaatccatggttttgggacgtttcctgagtcagattcgcgaaaaactgattcagagaatttaccgcgggatcgagccgactttgccaaac
tggttcgcggtcacaaagaccagaaatggcgccggaggcgggaacaaggtggtggatgagtgctacatccccaattacttgctccccaaaacccagcct
gagctccagtgggcgtggactaatatggaacagtatttaagcgcctgtttgaatctcacggagcgtaaacggttggtggcgcagcatctgacgcacgtg
tcgcagacgcaggagcagaacaaagagaatcagaatcccaattctgatgcgccggtgatcagatcaaaaacttcagccaggtacatggagctggtcggg
tggctcgtggacaaggggattacctcggagaagcagtggatccaggaggaccaggcctcatacatctccttcaatgcggcctccaactcgcggtcccaa
atcaaggctgccttggacaatgcgggaaagattatgagcctgactaaaaccgcccccgactacctggtgggccagcagcccgtggaggacatttccagc
aatcggatttataaaattttggaactaaacgggtacgatccccaatatgcggcttccgtctttctgggatgggccacgaaaaagttcggcaagaggaac
accatctggctgtttgggcctgcaactaccgggaagaccaacatcgcggaggccatagcccacactgtgcccttctacgggtgcgtaaactggaccaat
gagaactttcccttcaacgactgtgtcgacaagatggtgatctggtgggaggaggggaagatgaccgccaaggtcgtggagtcggccaaagccattctc
ggaggaagcaaggtgcgcgtggaccagaaatgcaagtcctcggcccagatagacccgactcccgtgatcgtcacctccaacaccaacatgtgcgccgtg
attgacgggaactcaacgaccttcgaacaccagcagccgttgcaagaccggatgttcaaatttgaactcacccgccgtctggatcatgactttgggaag
gtcaccaagcaggaagtcaaagactttttccggtgggcaaaggatcacgtggttgaggtggagcatgaattctacgtcaaaaagggtggagccaagaaa
agacccgcccccagtgacgcagatataagtgagcccaaacgggtgcgcgagtcagttgcgcagccatcgacgtcagacgcggaagcttcgatcaactac
gcagacaggtaccaaaacaaatgttctcgtcacgtgggcatgaatctgatgctgtttccctgcagacaatgcgagagaatgaatcagaattcaaatatc
tgcttcactcacggacagaaagactgtttagagtgctttcccgtgtcagaatctcaacccgtttctgtcgtcaaaaaggcgtatcagaaactgtgctac
attcatcatatcatgggaaaggtgccagacgcttgcactgcctgcgatctggtcaatgtggatttggatgactgcatctttgaacaataaatgatttaa
atcaggtatggctgccgatggttatcttccagattggctcgaggacactctctctgaaggaataagacagtggtggaagctcaaacctggcccaccacc
accaaagcccgcagagcggcataaggacgacagcaggggtcttgtgcttcctgggtacaagtacctcggacccttcaacggactcgacaagggagagcc
ggtcaacgaggcagacgccgcggccctcgagcacgacaaagcctacgaccggcagctcgacagcggagacaacccgtacctcaagtacaaccacgccga
cgcggagtttcaggagcgccttaaagaagatacgtcttttgggggcaacctcggacgagcagtcttccaggcgaaaaagagggttcttgaacctctggg
cctggttgaggaacctgttaagACCgctccgggaaaaaagaggccggtagagcactctcctgtggagccagactcctcctcgggaaccggaaaggcggg
ccagcagcctgcaagaaaaagattgaattttggtcagactggagacgcagactcagtacctgacccccagcctctcggacagccaccagcagccccctc
tggtctgggaactaatacgatggctacaggcagtggcgcaccaatggcagacaataacgagggcgccgacggagtgggtaattcctcgggaaattggca
ttgcgattccacatggatgggcgacagagtcatcaccaccagcacccgaacctgggccctgcccacctacaacaaccacctctacaaacaaatttccag
ccaatcaggagcctcgaacgacaatcactactttggctacagcaccccttgggggtattttgacttcaacagattccactgccacttttcaccacgtga
ctggcaaagactcatcaacaacaactggggattccgacccaagagactcaacttcaagctctttaacattcaagtcaaagaggtcacgcagaatgacgg
tacgacgacgattgccaataaccttaccagcacggttcaggtgtttactgactcggagtaccagctcccgtacgtcctcggctcggcgcatcaaggatg
cctcccgccgttcccagcagacgtcttcatggtgccacagtatggatacctcaccctgaacaacgggagtcaggcagtaggacgctcttcattttactg
cctggagtactttccttctcagatgctgcgtaccggaaacaactttaccttcagctacacttttgaggacgttcctttccacagcagctacgctcacag
ccagagtctggaccgtctcatgaatcctctcatcgaccagtacctgtattacttgagcagaacaaacactccaagtggaaccaccacgcagtcaaggct
tcagttttctcaggccggagcgagtgacattcgggaccagtctaggaactggcttcctggaccctgttaccgccagcagcgagtatcaaagacatctgc
ggataacaacaacagtgaatactcgtggactggagctaccaagtaccacctcaatggcagagactctctggtgaatccgggcccggccatggcaagcca
caaggacgatgaagaaaagttttttcctcagagcggggttctcatctttgggaagcaaggctcagagaaaacaaatgtggacattgaaaaggtcatgat
tacagacgaagaggaaatcaggacaaccaatcccgtggctacggagcagtatggttctgtatctaccaacctccagagaggcaacagacaagcagctac
cgcagatgtcaacacacaaggcgttcttccaggcatggtctggcaggacagagatgtgtaccttcaggggcccatctgggcaaagattccacacacgga
cggacattttcacccctctcccctcatgggggattcggacttaaacaccctcctccacagattctcatcaagaacaccccggtacctgcgaatccttcg
accaccttcagtgcggcaaagtttgcttccttcatcacacagtactccacgggacaggtcagcgtggagatcgagtgggagctgcagaaggaaaacagc
aaacgctggaatcccgaaattcagtacacttccaactacaacaagtctgttaatgtggactttactgtggacactaatggcgtgtattcagagcctcgc
cccattggcaccagatacctgactcgtaatctgtaattgcttgttaatcaataaaccgtttaattcgtttcagttgaactttggtctctgcgtatttct
ttcttatctagtttccatggctacgtagataagtagcatggcgggttaatcattaactacagcccgggcgtttaaacagcgggcggaggggtggagtcg
tgacgtgaattacgtcatagggttagggaggtcctgtattagaggtcacgtgagtgttttgcgacattttgcgacaccatgtggtctcgctgggggggg
gggcccgagtgagcacgcagggtctccattttgaagcgggaggtttgaacgagcgctggcgcgctcactggccgtcgttttacaacgtcgtgactggga
aaaccctggcgttacccaacttaatcgccttgcagcacatccccctttcgccagctggcgtaatagcgaagaggcccgcaccgatcgcccttcccatgc
atcggccgcaaatacctgcaggatccgttttgcgctgcttcgcgatgtacgggccagatatacgcgttgacattgattattgactagttattaatagta
atcaattacggggtcattagttcatagcccatatatggagttccgcgttacataacttacggtaaatggcccgcctggctgaccgcccaacgacccccg
cccattgacgtcaataatgacgtatgttcccatagtaacgccaatagggactttccattgacgtcaatgggggactatttacggtaaactgcccacttg
gcagtacatcaagtgtatcatatgccaagtacgccccctattgacgtcaatgacggtaaatggcccgcctggcattatgcccagtacatgaccttatgg
gactttcctacttggcagtacatctacgtattagtcatcgctattaccatggtgatgcggttttggcagtacatcaatgggcgtggatagcggtttgac
tcacggggatttccaagtctccaccccattgacgtcaatgggagtttgttttggcaccaaaatcaacgggactttccaaaatgtcgtaacaactccgcc
ccattgacgcaaatgggcggtaggcgtgtacggtgggaggtctatataagcagagctctctggctaactagagaacccactgcttactggcttatcgaa
attaatacgactcactatagggagacccaagctggctagcATGgctccgggaaaaaagaggccggtagagcactctcctgtggagccagactcctcctc
gggaaccggaaaggcgggccagcagcctgcaagaaaaagattgaattttggtcagactggagacgcagactcagtacctgacccccagcctctcggaca
gccaccagcagccccctctggtctCggaactaatacCCTCgctacaggcagtggcgcaccaCTCgcagacaataacgagggcgccgacggagtgggtaa
ttcctcgggaaattggcattgcgattccacatggCTCggcgacagagtcatcaccaccagcacccgaacctgggccctgcccacctacaacaaccacct
ctacaaacaaatttccagccaatcaggagcctcgaacgacaatcactactttggctacagcaccccttgggggtattttgacttcaacagattccactg
ccacttttcaccacgtgactggcaaagactcatcaacaacaactggggattccgacccaagagactcaacttcaagctctttaacattcaagtcaaaga
ggtcacgcagaatgacggtacgacgacgattgccaataaccttaccagcacggttcaggtgtttactgactcggagtaccagctcccgtacgtcctcgg
ctcggcgcatcaaggatgcctcccgccgttcccagcagacgtcttcatggtgccacagtatggatacctcaccctgaacaacgggagtcaggcagtagg
acgctcttcattttactgcctggagtactttccttctcagatgctgcgtaccggaaacaactttaccttcagctacacttttgaggacgttcctttcca
cagcagctacgctcacagccagagtctggaccgtctcatgaatcctctcatcgaccagtacctgtattacttgagcagaacaaacactccaagtggaac
cacc [symbol] cagtcaaggcttcagttttctcaggccggagcgagtgacattcgggaccagtctaggaactggcttcctggaccctgttaccgccagcagcg
agtatcaaagacatctgcggataacaacaacagtgaatactcgtggactggagctaccaagtaccacctcaatggcagagactctctggtgaatccggg
cccggccatggcaagccacaaggacgatgaagaaaagttttttcctcagagcggggttctcatctttgggaagcaaggctcagagaaaacaaatgtgga
cattgaaaaggtcatgattacagacgaagaggaaatcaggacaaccaatcccgtggctacggagcagtatggttctgtatctaccaacctccagagagg
caacagacaagcagctaccgcagatgtcaacacacaaggcgttcttccaggcatggtctggcaggacagagatgtgtaccttcaggggcccatctgggc
aaagattccacacacggacggacattttcacccctctcccctcatgggggattcggacttaaacaccctcctccacagattctcatcaagaacaccccg
gtacctgcgaatccttcgaccaccttcagtgcggcaaagtttgcttccttcatcacacagtactccacgggacaggtcagcgtggagatcgagtgggag
ctgcagaaggaaaacagcaaacgctggaatcccgaaattcagtacacttccaactacaacaagtctgttaatgtggactttactgtggacactaatggc
gtgtattcagagcctcgccccattggcaccagatacctgactcgtaatctgtaattgcttgttaatcaataaaccgtttaattcgtttcagttgaactt
tggtctctgcgtatttctttcttatctagtttccatggctacgtagataagtagcatggcgggttaatcattaactacagccctaggggtgcgagcgga
tcgagcagtgtcgatcactactggaccgcgagctgtgctgcgacccgtgatcttacggcattatacgtatgatcggtccacgatcagctagattatcta
gtcagcttgatgtcatagctgtttcctgaggctcaatactgaccatttaaatcatacctgacctccatagcagaaagtcaaaagcctccgaccggaggc
ttttgacttgatcggcacgtaagaggttccaactttcaccataatgaaataagatcactaccgggcgtattttttgagttatcgagattttcaggagct
aaggaagctaaaatgagccatattcaacgggaaacgtcttgcttgaagccgcgattaaattccaacatggatgctgatttatatgggtataaatgggct
cgcgataatgtcgggcaatcaggtgcgacaatctatcgattgtatgggaagcccgatgcgccagagttgtttctgaaacatggcaaaggtagcgttgcc
aatgatgttacagatgagatggtcaggctaaactggctgacggaatttatgcctcttccgaccatcaagcattttatccgtactcctgatgatgcatgg
ttactcaccactgcgatcccagggaaaacagcattccaggtattagaagaatatcctgattcaggtgaaaatattgttgatgcgctggcagtgttcctg
cgccggttgcattcgattcctgtttgtaattgtccttttaacggcgatcgcgtatttcgtctcgctcaggcgcaatcacgaatgaataacggtttggtt
ggtgcgagtgattttgatgacgagcgtaatggctggcctgttgaacaagtctggaaagaaatgcataaactcttgccattctcaccggattcagtcgtc
actcatggtgatttctcacttgataaccttatttttgacgaggggaaattaataggttgtattgatgttggacgagtcggaatcgcagaccgataccag
gatcttgccatcctatggaactgcctcggtgagttttctccttcattacagaaacggctttttcaaaaatatggtattgataatcctgatatgaataaa
ttgcagtttcacttgatgctcgatgagtttttctaatgaggacctaaatgtaatcacctggctcaccttcgggtgggcctttctgcgttgctggcgttt
ttccataggctccgcccccctgacgagcatcacaaaaatcgatgctcaagtcagaggtggcgaaacccgacaggactataaagataccaggcgtttccc
cctggaagctccctcgtgcgctctcctgttccgaccctgccgcttaccggatacctgtccgcctttctcccttcgggaagcgtggcgctttctcatagc
tcacgctgtaggtatctcagttcggtgtaggtcgttcgctccaagctgggctgtgtgcacgaaccccccgttcagcccgaccgctgcgccttatccggt
aactatcgtcttgagtccaacccggtaagacacgacttatcgccactggcagcagccactggtaacaggattagcagagcgaggtatgtaggcggtgct
acagagttcttgaagtggtggcctaactacggctacactagaagaacagtatttggtatctgcgctctgctgaagccagttacctcggaaaaagagttg
gtagctcttgatccggcaaacaaaccaccgctggtagcggtggtttttttgtttgcaagcagcagattacgcgcagaaaaaaaggatctcaagaagatc
ctttgattttctaccgaagaaaggccca

What is claimed is:

1. A virus-ribonucleoprotein conjugate (VRC).

2. Said VRC of claim 1 comprising a covalent guide RNA-virus conjugate.

3. Said guide RNA-virus conjugate of claim 2.

4. Said guide RNA-virus conjugate of claim 3, wherein the virus and the guide RNA are joined by a stable covalent linker.

5. Said guide RNA-virus conjugate of claim 3, wherein the virus and the guide RNA are joined by a cleavable covalent linker.

6. Said guide RNA-virus conjugate of claim 3, wherein the virus of is an AAV.

7. Said guide RNA-virus conjugate of claim 3, wherein capsid of said virus is modified with cell-targeting ligands, peptides, aptamers, or PEG.

8. Said guide RNA-virus conjugate of claim 3, wherein the virus provides a donor DNA template comprising a gene editing sequence flanked by two homology arms.

9. Said guide RNA-virus conjugate of claim 3, wherein the virus carries one or more transgenes.

10. Said guide RNA-virus conjugate of claim 3, wherein the guide RNA is bound by an RNA-guided endonuclease.

11. Said guide RNA-virus conjugate of claim 3, wherein the guide RNA is bound by a Cas protein.

12. Said guide RNA-virus conjugate of claim 11, wherein the Cas protein is a Cas-effector fusion protein.

13. Said guide RNA-virus conjugate of claim 12, wherein the fusion protein is delivered to targeted cells as an mRNA, DNA, plasmid or a viral vector.

14. Said guide RNA-virus conjugate of claim 12, wherein the effector protein is a DNA polymerase.

15. Said guide RNA-virus conjugate of claim 3, wherein the guide RNA is an optionally modified sgRNA.

16. Said guide RNA-virus conjugate of claim 3, wherein the guide is an optionally modified dual guide RNA (crRNA and tracrRNA).

17. Said guide RNA-virus conjugate of claim 3, wherein the guide RNA is an optionally modified crRNA.

18. Said guide RNA-virus conjugate of claim 3, wherein the guide RNA contains one or more internal non-nucleotide linkers.

19. A guide RNA-viral capsid protein conjugate.

20. A composition comprising said guide RNA-viral capsid protein conjugate of claim 19.


SEQUENCE LISTING (21 sequences)

SEQ ID NO: 1 - RNA, 42 nt, synthetic construct
  misc_difference 1..20: note = n is a, c, g or u.
  source 1..42: mol_type = other RNA
nnnnnnnnnnnnnnnnnnnngttttagagctatgctgttttg

SEQ ID NO: 2 - RNA, 81 nt, synthetic construct
  source 1..81: mol_type = other RNA
ggaaccattcaaaacagcatagcaagttaaaataaggctagtccgttatcaacttgaaaaagtggcaccgagtcggtgctt

SEQ ID NO: 3 - RNA, 32 nt, synthetic construct
  source 1..32: mol_type = other RNA
  misc_feature 32: note = SEQ ID 3 is joined at its 3'-end to the 5'-end SEQ ID 4 by a non-nucleotide linker.
nnnnnnnnnnnnnnnnnnnngttttagagctn

SEQ ID NO: 4 - RNA, 62 nt, synthetic construct
  source 1..62: mol_type = other RNA
  misc_feature 1: note = SEQ ID 4 is joined at its 5'-end to the 3'-end SEQ ID 3 by a non-nucleotide linker.
nagcaagttaaaataaggctagtccgttatcaacttgaaaaagtggcaccgagtcggtgctt

SEQ ID NO: 5 - RNA, 100 nt, synthetic construct
  source 1..100: mol_type = other RNA
  misc_feature 33..48: note = gaattcGAAAgaattc is EcoRI restriction site.
nnnnnnnnnnnnnnnnnnnngttttagagctagaaatagcaagttaaaataaggctagtccgttatcaacttgaaaaagtggcaccgagtcggtgctttt

SEQ ID NO: 6 - RNA, 38 nt, synthetic construct
  source 1..38: mol_type = other RNA
  misc_feature 1: note = SEQ ID 3 is joined at its 3'-end to the 5'-end SEQ ID 6 by a non-nucleotide linker.
  misc_feature 38: note = SEQ ID 6 is joined at its 3'-end to the 5'-end SEQ ID 7 by a non-nucleotide linker.
nagcaagttaaaataaggctagtccgttatcaacttga

SEQ ID NO: 7 - RNA, 24 nt, synthetic construct
  source 1..24: mol_type = other RNA
  misc_structure 1: note = SEQ ID 7 is joined at its 5'-end to the 3'-end SEQ ID 6 by a non-nucleotide linker.
aaaagtggcaccgagtcggtgctt

SEQ ID NO: 8 - RNA, 32 nt, synthetic construct
  source 1..32: mol_type = other RNA
  misc_feature 32: note = SEQ ID 8 is joined at its 3'-end to the 5'-end SEQ ID 9 by an aptamer nucleotide linker.
nnnnnnnnnnnnnnnnnnnngttttagagcta

SEQ ID NO: 9 - RNA, 62 nt, synthetic construct
  source 1..62: mol_type = other RNA
  misc_feature 1: note = SEQ ID 9 is joined at its 5'-end to the 3'-end SEQ ID 9 by an aptamer nucleotide linker.
tagcaagttaaaataaggctagtccgttatcaacttgaaaaagtggcaccgagtcggtgctt

SEQ ID NO: 10 - RNA, 32 nt, synthetic construct; identical to SEQ ID NO: 8
  source 1..32: mol_type = other RNA
  misc_feature 32: note = SEQ ID 10 is joined at its 3'-end to the 5'-end SEQ ID 11 by an aptamer nucleotide linker.

SEQ ID NO: 11 - RNA, 37 nt, synthetic construct
  source 1..37: mol_type = other RNA
  misc_feature 1: note = SEQ ID 11 is joined at its 5'-end to the 3'-end SEQ ID 10 by an aptamer nucleotide linker.
  misc_feature 37: note = SEQ ID 11 is joined at its 3'-end to the 5'-end SEQ ID 12 by an aptamer nucleotide linker.
tagcaagttaaaataaggctagtccgttatcaacttg

SEQ ID NO: 12 - RNA, 24 nt, synthetic construct
  source 1..24: mol_type = other RNA
  misc_feature 1: note = SEQ ID 12 is joined at its 5'-end to the 3'-end SEQ ID 11 by an aptamer nucleotide linker.
aaaagtggcaccgagtcggtgctt

SEQ ID NO: 13 - RNA, 36 nt, synthetic construct
  source 1..36: mol_type = other RNA
nnnnnnnnnnnnnnnnnnnngttttagagctatgct

SEQ ID NO: 14 - RNA, 66 nt, synthetic construct
  source 1..66: mol_type = other RNA
agcatagcaagttaaaataaggctagtccgttatcaacttgaaaaagtggcaccgagtcggtgctt

SEQ ID NO: 15 - RNA, 31 nt, synthetic construct
  source 1..31: mol_type = other RNA
  misc_feature 1: note = 1 is joined to the 5'-end of SEQ ID NO 16 at its 3'-position via a non-nucleotide linker.
ggtgggttaagnnnnnnnnnnnnnnnnnnnn

SEQ ID NO: 16 - RNA, 15 nt, synthetic construct
  source 1..15: mol_type = other RNA
  misc_feature 15: note = 3'-End OF SEQ ID NO 16 is joined to 5'-end of SEQ ID NO 15 via a non-nucleotide linker.
ggaagggaaccctcc

SEQ ID NO: 17 - RNA, 32 nt, synthetic construct
  source 1..32: mol_type = other RNA
  misc_feature 32: note = 3'-end is modified by a tethered azido group.
ggggccactagggacaggatgttttagagcta

SEQ ID NO: 18 - RNA, 63 nt, synthetic construct
  source 1..63: mol_type = other RNA
  misc_feature 1: note = 5'-end is modified by a tethered alkynyl group. 3'-end is modified by a tethered amino group or azido group.
tagcaagttaaaataaggctagtccgttatcaacttgaaaaagtggcaccgagtcggtgcttt

SEQ ID NO: 19 - RNA, 43 nt, synthetic construct
  source 1..43: mol_type = other RNA
  misc_feature 43: note = 3'-end is covalently joined to VP1/VP2 via an non-nucleotide linker at ncAA.
taatttctactgttgtagatnnnnnnnnnnnnnnnnnnnnnnn

SEQ ID NO: 20 - DNA, 9743 bp, synthetic construct
  source 1..9743: mol_type = other DNA
cccgtgtaaaacgacggccagtttatctagtcagcttgattctagctgatcgtggaccggaaggtgagccagtgagttgattgcagtccagttacgctggagtctgaggctcgtcctgaatgatatgcgaccgccggagggttgcgtttgagacgggcgacagatccagtcgcgctgctctcgtcgatccgctagggcggccgctctagaactagtggatcccccggaagatcagaagttcctattccgaagttcctattctctagaaagtataggaacttctgatctgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtctcgctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggtctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccatgcatcggccgcaaatacctgcaggatccgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagaccgctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctcggaactaataccctcgctacaggcagtggcgcaccactcgcagacaataacgagggcgccgacggagtgggtaattcctcgggaaattggcattgcgattccacatggctcggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccacctagcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacagccctaggggtgcgagcggatcgagcagtgtcgatcactactggaccgcgagctgtgctgcgacccgtgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagccatattcaacgggaaacgtcttgcttgaagccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataaactcttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaatgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggccca

SEQ ID NO: 21 - DNA, 9333 bp, synthetic construct
  source 1..9333: mol_type = other DNA
cccgtgtaaaacgacggccagtttatctagtcagcttgattctagctgatcgtggaccggaaggtgagccagtgagttgattgcagtccagttacgctggagtctgaggctcgtcctgaatgatatgcgaccgccggagggttgcgtttgagacgggcgacagatccagtcgcgctgctctcgtcgatccgctagggcggccgctctagaactagtggatcccccggaagatcagaagttcctattccgaagttcctattctctagaaagtataggaacttctgatctgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagaccgctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccatgcatcggccgcaaatacctgcaggatccgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcatggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctcggaactaataccctcgctacaggcagtggcgcaccactcgcagacaataacgagggcgccgacggagtgggtaattcctcgggaaattggcattgcgattccacatggctcggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccacctagcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacagccctaggggtgcgagcggatcgagcagtgtcgatcactactggaccgcgagctgtgctgcgacccgtgatcttacggcattatacgtatgatcggtccacgatcagctagattatctagtcagcttgatgtcatagctgtttcctgaggctcaatactgaccatttaaatcatacctgacctccatagcagaaagtcaaaagcctccgaccggaggcttttgacttgatcggcacgtaagaggttccaactttcaccataatgaaataagatcactaccgggcgtattttttgagttatcgagattttcaggagctaaggaagctaaaatgagccatattcaacgggaaacgtcttgcttgaagccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcaggctaaactggctgacggaatttatgcctcttccgaccatcaagcattttatccgtactcctgatgatgcatggttactcaccactgcgatcccagggaaaacagcattccaggtattagaagaatatcctgattcaggtgaaaatattgttgatgcgctggcagtgttcctgcgccggttgcattcgattcctgtttgtaattgtccttttaacggcgatcgcgtatttcgtctcgctcaggcgcaatcacgaatgaataacggtttggttggtgcgagtgattttgatgacgagcgtaatggctggcctgttgaacaagtctggaaagaaatgcataaactcttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcacttgatgctcgatgagtttttctaatgaggacctaaatgtaatcacctggctcaccttcgggtgggcctttctgcgttgctggcgtttttccataggctccgcccccctgacgagcatcacaaaaatcgatgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttacctcggaaaaagagttggtagctcttgatccggcaaacaaaccaccgctggtagcggtggtttttttgtttgcaagcagcagattacgcgcagaaaaaaaggatctcaagaagatcctttgattttctaccgaagaaaggccca